SEQUENCE LISTING (136 sequences)

SEQ ID NO: 1 - DNA, 21 bp, synthetic construct
  source 1..21: mol_type = other DNA
  misc_feature 1..19: note = RNA
  misc_feature 20..21: note = DNA
ggttcttctggactatcaatt

SEQ ID NO: 2 - DNA, 21 bp, synthetic construct
  source 1..21: mol_type = other DNA
  misc_feature 1..19: note = RNA
  misc_feature 20..21: note = DNA
ttgatagtccagaagaacctt

SEQ ID NO: 3 - DNA, 21 bp, synthetic construct
  source 1..21: mol_type = other DNA
  misc_feature 1..19: note = RNA
  misc_feature 20..21: note = DNA
cgcttgggactctctcgtctt

SEQ ID NO: 4 - DNA, 21 bp, synthetic construct
  source 1..21: mol_type = other DNA
  misc_feature 1..19: note = RNA
  misc_feature 20..21: note = DNA
gacgagagagtcccaagcgtt

SEQ ID NO: 5 - DNA, 21 bp, synthetic construct
  source 1..21: mol_type = other DNA
  misc_feature 1..19: note = RNA
  misc_feature 20..21: note = DNA
cgcagtccccaacctccaatt

SEQ ID NO: 6 - DNA, 21 bp, synthetic construct
  source 1..21: mol_type = other DNA
  misc_feature 1..19: note = RNA
  misc_feature 20..21: note = DNA
ttggaggttggggactgcgtt

SEQ ID NO: 7 - DNA, 21 bp, synthetic construct
  source 1..21: mol_type = other DNA
  misc_feature 1..19: note = RNA
  misc_feature 20..21: note = DNA
ccaacctcctgtcctccaatt

SEQ ID NO: 8 - DNA, 21 bp, synthetic construct
  source 1..21: mol_type = other DNA
  misc_feature 1..19: note = RNA
  misc_feature 20..21: note = DNA
ttggaggacaggaggttggtt

SEQ ID NO: 9 - DNA, 21 bp, synthetic construct
  source 1..21: mol_type = other DNA
  misc_feature 1..19: note = RNA
  misc_feature 20..21: note = DNA
gtcctggttatcgctggattt

SEQ ID NO: 10 - DNA, 21 bp, synthetic construct
  source 1..21: mol_type = other DNA
  misc_feature 1..19: note = RNA
  misc_feature 20..21: note = DNA
atccagcgataaccaggactt

SEQ ID NO: 11 - DNA, 21 bp, synthetic construct
  source 1..21: mol_type = other DNA
  misc_feature 1..19: note = RNA
  misc_feature 20..21: note = DNA
cctcttcatcctgctgctatt

SEQ ID NO: 12 - DNA, 21 bp, synthetic construct
  source 1..21: mol_type = other DNA
  misc_feature 1..19: note = RNA
  misc_feature 20..21: note = DNA
tagcagcaggatgaagaggtt

SEQ ID NO: 13 - DNA, 21 bp, synthetic construct
  source 1..21: mol_type = other DNA
  misc_feature 1..19: note = RNA
  misc_feature 20..21: note = DNA
ctgctgctatgcctcatcttt

SEQ ID NO: 14 - DNA, 21 bp, synthetic construct
  source 1..21: mol_type = other DNA
  misc_feature 1..19: note = RNA
  misc_feature 20..21: note = DNA
agatgaggcatagcagcagtt

SEQ ID NO: 15 - DNA, 21 bp, synthetic construct
  source 1..21: mol_type = other DNA
  misc_feature 1..19: note = RNA
  misc_feature 20..21: note = DNA
gacgcaacccccactggcttt

SEQ ID NO: 16 - DNA, 21 bp, synthetic construct
  source 1..21: mol_type = other DNA
  misc_feature 1..19: note = RNA
  misc_feature 20..21: note = DNA
agccagtgggggttgcgtctt

SEQ ID NO: 17 - DNA, 21 bp, synthetic construct
  source 1..21: mol_type = other DNA
  misc_feature 1..19: note = RNA
  misc_feature 20..21: note = DNA
ctctcgtccccttctccgttt

SEQ ID NO: 18 - DNA, 21 bp, synthetic construct
  source 1..21: mol_type = other DNA
  misc_feature 1..19: note = RNA
  misc_feature 20..21: note = DNA
acggagaaggggacgagagtt

SEQ ID NO: 19 - DNA, 21 bp, synthetic construct
  source 1..21: mol_type = other DNA
  misc_feature 1..19: note = RNA
  misc_feature 20..21: note = DNA
gggctttcccccactgttttt

SEQ ID NO: 20 - DNA, 21 bp, synthetic construct
  source 1..21: mol_type = other DNA
  misc_feature 1..19: note = RNA
  misc_feature 20..21: note = DNA
aaacagtgggggaaagccctt

SEQ ID NO: 21 - DNA, 21 bp, synthetic construct
  source 1..21: mol_type = other DNA
  misc_feature 1..19: note = RNA
  misc_feature 20..21: note = DNA
cctattgattggaaagtattt

SEQ ID NO: 22 - DNA, 21 bp, synthetic construct
  source 1..21: mol_type = other DNA
  misc_feature 1..19: note = RNA
  misc_feature 20..21: note = DNA
atactttccaatcaataggtt

SEQ ID NO: 23 - RNA, 25 nt, synthetic construct
  source 1..25: mol_type = other RNA
gcagtccccaacctccaatcactca

SEQ ID NO: 24 - RNA, 25 nt, synthetic construct
  source 1..25: mol_type = other RNA
tgagtgattggaggttggggactgc

SEQ ID NO: 25 - RNA, 25 nt, synthetic construct
  source 1..25: mol_type = other RNA
gtccccaacctccaatcactcacca

SEQ ID NO: 26 - RNA, 25 nt, synthetic construct
  source 1..25: mol_type = other RNA
tggtgagtgattggaggttggggac

SEQ ID NO: 27 - RNA, 25 nt, synthetic construct
  source 1..25: mol_type = other RNA
ccaacctccaatcactcaccaacct

SEQ ID NO: 28 - RNA, 25 nt, synthetic construct
  source 1..25: mol_type = other RNA
aggttggtgagtgattggaggttgg

SEQ ID NO: 29 - RNA, 25 nt, synthetic construct
  source 1..25: mol_type = other RNA
gtcctggttatcgctggatgtgtct

SEQ ID NO: 30 - RNA, 25 nt, synthetic construct
  source 1..25: mol_type = other RNA
agacacatccagcgataaccaggac

SEQ ID NO: 31 - RNA, 25 nt, synthetic construct
  source 1..25: mol_type = other RNA
ctcttcatcctgctgctatgcctca

SEQ ID NO: 32 - RNA, 25 nt, synthetic construct
  source 1..25: mol_type = other RNA
tgaggcatagcagcaggatgaagag

SEQ ID NO: 33 - RNA, 25 nt, synthetic construct
  source 1..25: mol_type = other RNA
gttgcccgtttgtcctctaattcca

SEQ ID NO: 34 - RNA, 25 nt, synthetic construct
  source 1..25: mol_type = other RNA
tggaattagaggacaaacgggcaac

SEQ ID NO: 35 - RNA, 25 nt, synthetic construct
  source 1..25: mol_type = other RNA
gtgcacttcgcttcacctctgcacg

SEQ ID NO: 36 - RNA, 25 nt, synthetic construct
  source 1..25: mol_type = other RNA
cgtgcagaggtgaagcgaagtgcac

SEQ ID NO: 37 - DNA, 21 bp, synthetic construct
  source 1..21: mol_type = other DNA
  misc_feature 1..19: note = RNA
  misc_feature 20..21: note = DNA
cctgctggtggctccagtttt

SEQ ID NO: 38 - DNA, 21 bp, synthetic construct
  source 1..21: mol_type = other DNA
  misc_feature 1..19: note = RNA
  misc_feature 20..21: note = DNA
aactggagccaccagcaggtt

SEQ ID NO: 39 - DNA, 21 bp, synthetic construct
  source 1..21: mol_type = other DNA
  misc_feature 1..19: note = RNA
  misc_feature 20..21: note = DNA
cagcaatgtcaacgaccgatt

SEQ ID NO: 40 - DNA, 21 bp, synthetic construct
  source 1..21: mol_type = other DNA
  misc_feature 1..19: note = RNA
  misc_feature 20..21: note = DNA
tcggtcgttgacattgctgtt

SEQ ID NO: 41 - DNA, 21 bp, synthetic construct
  source 1..21: mol_type = other DNA
  misc_feature 1..19: note = RNA
  misc_feature 20..21: note = DNA
ccttggactcataaggtggtt

SEQ ID NO: 42 - DNA, 21 bp, synthetic construct
  source 1..21: mol_type = other DNA
  misc_feature 1..19: note = RNA
  misc_feature 20..21: note = DNA
ccaccttatgagtccaaggtt

SEQ ID NO: 43 - DNA, 21 bp, synthetic construct
  source 1..21: mol_type = other DNA
  misc_feature 1..19: note = RNA
  misc_feature 20..21: note = DNA
ggtggagccctcaggctcatt

SEQ ID NO: 44 - DNA, 21 bp, synthetic construct
  source 1..21: mol_type = other DNA
  misc_feature 1..19: note = RNA
  misc_feature 20..21: note = DNA
tgagcctgagggctccacctt

SEQ ID NO: 45 - DNA, 21 bp, synthetic construct
  source 1..21: mol_type = other DNA
  misc_feature 1..19: note = RNA
  misc_feature 20..21: note = DNA
cagtcaggaaggcagcctatt

SEQ ID NO: 46 - DNA, 21 bp, synthetic construct
  source 1..21: mol_type = other DNA
  misc_feature 1..19: note = RNA
  misc_feature 20..21: note = DNA
taggctgccttcctgactgtt

SEQ ID NO: 47 - DNA, 21 bp, synthetic construct
  source 1..21: mol_type = other DNA
  misc_feature 1..19: note = RNA
  misc_feature 20..21: note = DNA
ggtggctccagttcaggaatt

SEQ ID NO: 48 - DNA, 21 bp, synthetic construct
  source 1..21: mol_type = other DNA
  misc_feature 1..19: note = RNA
  misc_feature 20..21: note = DNA
ttcctgaactggagccacctt

SEQ ID NO: 49 - DNA, 21 bp, synthetic construct
  source 1..21: mol_type = other DNA
  misc_feature 1..19: note = RNA
  misc_feature 20..21: note = DNA
gttgacaagaatcctcacatt

SEQ ID NO: 50 - DNA, 21 bp, synthetic construct
  source 1..21: mol_type = other DNA
  misc_feature 1..19: note = RNA
  misc_feature 20..21: note = DNA
tgtgaggattcttgtcaactt

SEQ ID NO: 51 - DNA, 21 bp, synthetic construct
  source 1..21: mol_type = other DNA
  misc_feature 1..19: note = RNA
  misc_feature 20..21: note = DNA
gacaagaatcctcacaatatt

SEQ ID NO: 52 - DNA, 21 bp, synthetic construct
  source 1..21: mol_type = other DNA
  misc_feature 1..19: note = RNA
  misc_feature 20..21: note = DNA
tattgtgaggattcttgtctt

SEQ ID NO: 53 - DNA, 21 bp, synthetic construct
  source 1..21: mol_type = other DNA
  misc_feature 1..19: note = RNA
  misc_feature 20..21: note = DNA
ctagactcgtggtggactttt

SEQ ID NO: 54 - DNA, 21 bp, synthetic construct
  source 1..21: mol_type = other DNA
  misc_feature 1..19: note = RNA
  misc_feature 20..21: note = DNA
aagtccaccacgagtctagtt

SEQ ID NO: 55 - DNA, 21 bp, synthetic construct
  source 1..21: mol_type = other DNA
  misc_feature 1..19: note = RNA
  misc_feature 20..21: note = DNA
gagattaggttaaaggtcttt

SEQ ID NO: 56 - DNA, 21 bp, synthetic construct
  source 1..21: mol_type = other DNA
  misc_feature 1..19: note = RNA
  misc_feature 20..21: note = DNA
agacctttaacctaatctctt

SEQ ID NO: 57 - DNA, 21 bp, synthetic construct
  source 1..21: mol_type = other DNA
  misc_feature 1..19: note = RNA
  misc_feature 20..21: note = DNA
gcataaattggtctgcgcatt

SEQ ID NO: 58 - DNA, 21 bp, synthetic construct
  source 1..21: mol_type = other DNA
  misc_feature 1..19: note = RNA
  misc_feature 20..21: note = DNA
tgcgcagaccaatttatgctt

SEQ ID NO: 59 - DNA, 21 bp, synthetic construct
  source 1..21: mol_type = other DNA
  misc_feature 1..19: note = RNA
  misc_feature 20..21: note = DNA
gcgcaccagcaccatgcaatt

SEQ ID NO: 60 - DNA, 21 bp, synthetic construct
  source 1..21: mol_type = other DNA
  misc_feature 1..19: note = RNA
  misc_feature 20..21: note = DNA
ttgcatggtgctggtgcgctt

SEQ ID NO: 61 - DNA, 21 bp, synthetic construct
  source 1..21: mol_type = other DNA
  misc_feature 1..19: note = RNA
  misc_feature 20..21: note = DNA
cctctgcctaatcatctcttt

SEQ ID NO: 62 - DNA, 21 bp, synthetic construct
  source 1..21: mol_type = other DNA
  misc_feature 1..19: note = RNA
  misc_feature 20..21: note = DNA
agagatgattaggcagaggtt

SEQ ID NO: 63 - DNA, 21 bp, synthetic construct
  source 1..21: mol_type = other DNA
  misc_feature 1..19: note = RNA
  misc_feature 20..21: note = DNA
cacttccggaaactactgttt

SEQ ID NO: 64 - DNA, 21 bp, synthetic construct
  source 1..21: mol_type = other DNA
  misc_feature 1..19: note = RNA
  misc_feature 20..21: note = DNA
acagtagtttccggaagtgtt

SEQ ID NO: 65 - DNA, 21 bp, synthetic construct
  source 1..21: mol_type = other DNA
  misc_feature 1..19: note = RNA
  misc_feature 20..21: note = DNA
ccctcaggctcagggcatatt

SEQ ID NO: 66 - DNA, 21 bp, synthetic construct
  source 1..21: mol_type = other DNA
  misc_feature 1..19: note = RNA
  misc_feature 20..21: note = DNA
tatgccctgagcctgagggtt

SEQ ID NO: 67 - DNA, 21 bp, synthetic construct
  source 1..21: mol_type = other DNA
  misc_feature 1..19: note = RNA
  misc_feature 20..21: note = DNA
cgaggcaggtcccctagaatt

SEQ ID NO: 68 - DNA, 21 bp, synthetic construct
  source 1..21: mol_type = other DNA
  misc_feature 1..19: note = RNA
  misc_feature 20..21: note = DNA
ttctaggggacctgcctcgtt

SEQ ID NO: 69 - DNA, 21 bp, synthetic construct
  source 1..21: mol_type = other DNA
  misc_feature 1..19: note = RNA
  misc_feature 20..21: note = DNA
gggaatctcaatgttagtatt

SEQ ID NO: 70 - DNA, 21 bp, synthetic construct
  source 1..21: mol_type = other DNA
  misc_feature 1..19: note = RNA
  misc_feature 20..21: note = DNA
tactaacattgagattccctt

SEQ ID NO: 71 - DNA, 21 bp, synthetic construct
  source 1..21: mol_type = other DNA
  misc_feature 1..19: note = RNA
  misc_feature 20..21: note = DNA
ggaatctcaatgttagtattt

SEQ ID NO: 72 - DNA, 21 bp, synthetic construct
  source 1..21: mol_type = other DNA
  misc_feature 1..19: note = RNA
  misc_feature 20..21: note = DNA
atactaacattgagattcctt

SEQ ID NO: 73 - DNA, 21 bp, synthetic construct
  source 1..21: mol_type = other DNA
  misc_feature 1..19: note = RNA
  misc_feature 20..21: note = DNA
catcacatcaggattcctatt

SEQ ID NO: 74 - DNA, 21 bp, synthetic construct
  source 1..21: mol_type = other DNA
  misc_feature 1..19: note = RNA
  misc_feature 20..21: note = DNA
taggaatcctgatgtgatgtt

SEQ ID NO: 75 - RNA, 25 nt, synthetic construct
  source 1..25: mol_type = other RNA
gaacatggagaacatcacatcagga

SEQ ID NO: 76 - RNA, 25 nt, synthetic construct
  source 1..25: mol_type = other RNA
tcctgatgtgatgttctccatgttc

SEQ ID NO: 77 - RNA, 25 nt, synthetic construct
  source 1..25: mol_type = other RNA
ggagaacatcacatcaggattccta

SEQ ID NO: 78 - RNA, 25 nt, synthetic construct
  source 1..25: mol_type = other RNA
taggaatcctgatgtgatgttctcc

SEQ ID NO: 79 - RNA, 25 nt, synthetic construct
  source 1..25: mol_type = other RNA
gaacatcacatcaggattcctagga

SEQ ID NO: 80 - RNA, 25 nt, synthetic construct
  source 1..25: mol_type = other RNA
tcctaggaatcctgatgtgatgttc

SEQ ID NO: 81 - RNA, 25 nt, synthetic construct
  source 1..25: mol_type = other RNA
cttgttgacaagaatcctcacaata

SEQ ID NO: 82 - RNA, 25 nt, synthetic construct
  source 1..25: mol_type = other RNA
tattgtgaggattcttgtcaacaag

SEQ ID NO: 83 - RNA, 25 nt, synthetic construct
  source 1..25: mol_type = other RNA
cagagtctagactcgtggtggactt

SEQ ID NO: 84 - RNA, 25 nt, synthetic construct
  source 1..25: mol_type = other RNA
aagtccaccacgagtctagactctg

SEQ ID NO: 85 - RNA, 25 nt, synthetic construct
  source 1..25: mol_type = other RNA
gagtctagactcgtggtggacttct

SEQ ID NO: 86 - RNA, 25 nt, synthetic construct
  source 1..25: mol_type = other RNA
agaagtccaccacgagtctagactc

SEQ ID NO: 87 - RNA, 25 nt, synthetic construct
  source 1..25: mol_type = other RNA
gtctagactcgtggtggacttctct

SEQ ID NO: 88 - RNA, 25 nt, synthetic construct
  source 1..25: mol_type = other RNA
agagaagtccaccacgagtctagac

SEQ ID NO: 89 - RNA, 25 nt, synthetic construct
  source 1..25: mol_type = other RNA
gcatggagaccaccgtgaacgccca

SEQ ID NO: 90 - RNA, 25 nt, synthetic construct
  source 1..25: mol_type = other RNA
tgggcgttcacggtggtctccatgc

SEQ ID NO: 91 - RNA, 25 nt, synthetic construct
  source 1..25: mol_type = other RNA
ctgtaggcataaattggtctgcgca

SEQ ID NO: 92 - RNA, 25 nt, synthetic construct
  source 1..25: mol_type = other RNA
tgcgcagaccaatttatgcctacag

SEQ ID NO: 93 - RNA, 25 nt, synthetic construct
  source 1..25: mol_type = other RNA
gcataaattggtctgcgcaccagca

SEQ ID NO: 94 - RNA, 25 nt, synthetic construct
  source 1..25: mol_type = other RNA
tgctggtgcgcagaccaatttatgc

SEQ ID NO: 95 - RNA, 25 nt, synthetic construct
  source 1..25: mol_type = other RNA
gccttgggtggctttggggcatgga

SEQ ID NO: 96 - RNA, 25 nt, synthetic construct
  source 1..25: mol_type = other RNA
tccatgccccaaagccacccaaggc

SEQ ID NO: 97 - RNA, 25 nt, synthetic construct
  source 1..25: mol_type = other RNA
gaatttggagctactgtggagttac

SEQ ID NO: 98 - RNA, 25 nt, synthetic construct
  source 1..25: mol_type = other RNA
gtaactccacagtagctccaaattc

SEQ ID NO: 99 - RNA, 25 nt, synthetic construct
  source 1..25: mol_type = other RNA
ctccagttcaggaacagtaaaccct

SEQ ID NO: 100 - RNA, 25 nt, synthetic construct
  source 1..25: mol_type = other RNA
agggtttactgttcctgaactggag

SEQ ID NO: 101 - RNA, 25 nt, synthetic construct
  source 1..25: mol_type = other RNA
cctgagcattgctcacctcaccata

SEQ ID NO: 102 - RNA, 25 nt, synthetic construct
  source 1..25: mol_type = other RNA
tatggtgaggtgagcaatgctcagg

SEQ ID NO: 103 - RNA, 25 nt, synthetic construct
  source 1..25: mol_type = other RNA
ccaccaatcggcagtcaggaaggca

SEQ ID NO: 104 - RNA, 25 nt, synthetic construct
  source 1..25: mol_type = other RNA
tgccttcctgactgccgattggtgg

SEQ ID NO: 105 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  misc_feature 1..19: note = RNA
  misc_feature 20..21: note = DNA
cgcagtccccaacctccaatt

SEQ ID NO: 106 - DNA, 21 bp, synthetic construct
  source 1..21: mol_type = other DNA
  modified_base 1: mod_base = OTHER; note = Phosphorylated 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  misc_feature 1..19: note = RNA
  misc_feature 20..21: note = DNA
ttggaggttggggactgcgtt

SEQ ID NO: 107 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  misc_feature 1..19: note = RNA
  misc_feature 20..21: note = DNA
cctcttcatcctgctgctatt

SEQ ID NO: 108 - DNA, 21 bp, synthetic construct
  source 1..21: mol_type = other DNA
  modified_base 1: mod_base = OTHER; note = Phosphorylated 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  misc_feature 1..19: note = RNA
  misc_feature 20..21: note = DNA
tagcagcaggatgaagaggtt

SEQ ID NO: 109 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  misc_feature 1..19: note = RNA
  misc_feature 20..21: note = DNA
ctctcgtccccttctccgttt

SEQ ID NO: 110 - DNA, 21 bp, synthetic construct
  source 1..21: mol_type = other DNA
  modified_base 1: mod_base = OTHER; note = Phosphorylated 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  misc_feature 1..19: note = RNA
  misc_feature 20..21: note = DNA
acggagaaggggacgagagtt

SEQ ID NO: 111 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  misc_feature 1..19: note = RNA
  misc_feature 20..21: note = DNA
cctattgattggaaagtattt

SEQ ID NO: 112 - DNA, 21 bp, synthetic construct
  source 1..21: mol_type = other DNA
  modified_base 1: mod_base = OTHER; note = Phosphorylated 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  misc_feature 1..19: note = RNA
  misc_feature 20..21: note = DNA
atactttccaatcaataggtt

SEQ ID NO: 113 - DNA, 21 bp, synthetic construct
  source 1..21: mol_type = other DNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-fluoro modified nucleotide
  modified_base 9: mod_base = OTHER; note = 2'-fluoro modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  misc_feature 1..19: note = RNA
  misc_feature 20..21: note = DNA
gcataaattggtctgcgcatt

SEQ ID NO: 114 - DNA, 21 bp, synthetic construct
  source 1..21: mol_type = other DNA
  modified_base 1: mod_base = OTHER; note = Phosphorylated 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  misc_feature 1..19: note = RNA
  misc_feature 20..21: note = DNA
tgcgcagaccaatttatgctt

SEQ ID NO: 115 - RNA, 25 nt, synthetic construct
  source 1..25: mol_type = other RNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
  modified_base 22: mod_base = OTHER; note = 2'-O-methyl modified nucleotide
  modified_base 23: mod_base = OTHER; note = 2'-O-methyl modified nucleotide
  modified_base 24: mod_base = OTHER; note = 2'-O-methyl modified nucleotide
  modified_base 25: mod_base = OTHER; note = 2'-O-methyl modified nucleotide
ccaacctccaatcactcaccaacct

SEQ ID NO: 116 - RNA, 25 nt, synthetic construct
  source 1..25: mol_type = other RNA
  modified_base 1: mod_base = OTHER; note = Phosphorylated 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
  modified_base 22: mod_base = OTHER; note = 2'-O-methyl modified nucleotide
  modified_base 23: mod_base = OTHER; note = 2'-O-methyl modified nucleotide
  modified_base 24: mod_base = OTHER; note = 2'-O-methyl modified nucleotide
  modified_base 25: mod_base = OTHER; note = 2'-O-methyl modified nucleotide
aggttggtgagtgattggaggttgg

SEQ ID NO: 117 - RNA, 25 nt, synthetic construct
  source 1..25: mol_type = other RNA
  modified_base 1: mod_base = OTHER; note = 2'-O-methyl modified nucleotide
  modified_base 2: mod_base = OTHER; note = 2'-O-methyl modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-fluoro modified nucleotide
  modified_base 14: mod_base = OTHER; note = 2'-fluoro modified nucleotide
  modified_base 15: mod_base = OTHER; note = 2'-fluoro modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
  modified_base 22: mod_base = OTHER; note = 2'-O-methyl modified nucleotide
  modified_base 23: mod_base = OTHER; note = 2'-O-methyl modified nucleotide
  modified_base 24: mod_base = OTHER; note = 2'-O-methyl modified nucleotide
  modified_base 25: mod_base = OTHER; note = 2'-O-methyl modified nucleotide
ctgtaggcataaattggtctgcgca

SEQ ID NO: 118 - RNA, 25 nt, synthetic construct
  source 1..25: mol_type = other RNA
  modified_base 1: mod_base = OTHER; note = Phosphorylated 2'-O-methyl modified nucleotide
  modified_base 2: mod_base = OTHER; note = 2'-fluoro modified nucleotide
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-O-methyl modified nucleotide
  modified_base 8: mod_base = OTHER; note = 2'-O-methyl modified nucleotide
  modified_base 9: mod_base = OTHER; note = 2'-O-methyl modified nucleotide
  modified_base 10: mod_base = OTHER; note = 2'-O-methyl modified nucleotide
  modified_base 11: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide
  modified_base 22: mod_base = OTHER; note = 2'-O-methyl modified nucleotide
  modified_base 23: mod_base = OTHER; note = 2'-O-methyl modified nucleotide
  modified_base 24: mod_base = OTHER; note = 2'-O-methyl modified nucleotide
  modified_base 25: mod_base = OTHER; note = 2'-O-methyl modified nucleotide
tgcgcagaccaatttatgcctacag

SEQ ID NO: 119 - protein, 205 aa, synthetic construct
  VARIANT 1..200: note = This region may encompass 1-10 HnKm repeating units, wherein n is 1-10 and m is 1-10
  VARIANT 201..205: note = This region may encompass 1-5 residues
  VARIANT 201..205: note = Any amino acid
HHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKXXXXX

SEQ ID NO: 120 - protein, 4 aa, synthetic construct
HHHH

SEQ ID NO: 121 - protein, 4 aa, synthetic construct
HHHK

SEQ ID NO: 122 - protein, 37 aa, synthetic construct
  VARIANT 1..9: note = This region may be absent
  VARIANT 19..27: note = This region may be absent
  VARIANT 37: note = This residue may be attached to a hexyl group, a PEG group or another sequence
KHHHKHHHHKHHHKHHHKKHHHKHHHHKHHHKHHHKK

SEQ ID NO: 123 - protein, 4 aa, synthetic construct
SSSS

SEQ ID NO: 124 - protein, 5 aa, synthetic construct
SSSSS

SEQ ID NO: 125 - protein, 6 aa, synthetic construct
SSSSSS

SEQ ID NO: 126 - protein, 4 aa, synthetic construct
TTTT

SEQ ID NO: 127 - protein, 11 aa, synthetic construct
HHHKHHCKHHH

SEQ ID NO: 128 - protein, 11 aa, synthetic construct
HHHKHHCRHHH

SEQ ID NO: 129 - protein, 7 aa, synthetic construct
  VARIANT 6: note = H, K or R
HKHHCXH

SEQ ID NO: 130 - protein, 6 aa, synthetic construct
  VARIANT 5: note = K or R
HKHCXH

SEQ ID NO: 131 - protein, 6 aa, synthetic construct
HKHCKH

SEQ ID NO: 132 - protein, 7 aa, synthetic construct
  SITE 1: note = polyethylene glycol 4-Bis-NHS-(Trifluoroacetylamino )-hexyl-(2-cyanoethyl)-(N,N-diisopropyl)-phosphoramidite attached to side chain of lysine
  SITE 5: note = trivalent beta-N-Acetylgalactosamine polyethylene glycol maleimide modified residue
  VARIANT 6: note = K, R or H
KHHHCXH

SEQ ID NO: 133 - protein, 7 aa, synthetic construct
  SITE 1: note = Bis-polyethylene glycol 4-(Trifluoroacetylamino)-he xyl-(2-cyanoethyl)-(N,N-diisopropyl)-phosphoramidite attached to side chain of lysine
  SITE 5: note = trivalent beta-N-Acetylgalactosamine polyethylene glycol maleimide modified residue
  VARIANT 6: note = K, R or H
KHHHCXH

SEQ ID NO: 134 - protein, 7 aa, synthetic construct
  SITE 1: note = polyethylene glycol 4-azide-dibenzocyclooctyl-6-oxo -6-(dibenzo[b,f]azacyclooct-4-yn-1-yl)-caproic acid sulfo-N-hydroxysuccinimide ester, sodium salt-(Trifluoroac etylamino)-hexyl-(2-cyanoethyl)-(N,N-diisopropyl)-phosphor amidite attached to side chain of lysine
  SITE 5: note = trivalent beta-N-Acetylgalactosamine polyethylene glycol maleimide modified residue
KHHHCKH

SEQ ID NO: 135 - protein, 7 aa, synthetic construct
  SITE 1: note = polyethylene glycol 4-Bis-NHS-dibenzocyclooctyl-(Tr ifluoroacetylamino)-hexyl-(2-cyanoethyl)-(N,N-diisopropyl) -phosphoramidite attached to side chain of lysine
  SITE 5: note = trivalent beta-N-Acetylgalactosamine polyethylene glycol maleimide modified residue
  VARIANT 6: note = K, R or H
KHHHCXH

SEQ ID NO: 136 - protein, 210 aa, synthetic construct
  VARIANT 1..200: note = This region may encompass 1-10 HnKm repeating units, wherein n is 1-10 and m is 1-10
  VARIANT 201..205: note = This region may encompass 1-5 residues
  VARIANT 206..210: note = This region may encompass 1-5 residues
  VARIANT 201..210: note = Any amino acid
HHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKHHHHHHHHHHKKKKKKKKKKXXXXXXXXXX

(19) United States
(12) Patent Application Publication
(10) Pub. No.: US 2023/0392154 A1
(43) Pub. Date: Dec. 7, 2023

(54) TARGETED NUCLEIC ACID THERAPY FOR HEPATITIS B

(71) Applicant: Sirnaomics, Inc., Germantown, MD (US)

(72) Inventors: Xiaoyong LU, Germantown, MD (US); Patrick Y. LU, Germantown, MD (US); David M. EVANS, Germantown, MD (US); Danny TANG, Guangzhou (CN)

(21) Appl. No.: 18/179,123

(22) Filed: Mar. 6, 2023

Related U.S. Application Data
(63) Continuation of application No. PCT/IB22/00028, filed on Jan. 21, 2022.
(60) Provisional application No. 63/140,232, filed on Jan. 21, 2021.

(51) Int. Cl. C12N 15/113; A61P 31/20

(52) U.S. Cl. CPC C12N 15/1131; A61P 31/20; C12N 2310/14

(57) ABSTRACT

Pharmaceutical constructs containing a Peptide Docking Vehicle (PDoV) covalently linked to: (a) a targeting moiety; and (b) a first therapeutic nucleic acid, are provided, where the therapeutic nucleic acid inhibits replication of Hepatitis B virus (HBV). Methods of using the constructs for treating HBV in subjects also are provided.

### CROSS REFERENCE TO RELATED APPLICATIONS

This application is a continuation of international application PCT/IB2022/000028, filed Jan. 21, 2022, which claims the benefit of and priority to U.S. Provisional Patent Application No. 63/140,232, filed Jan. 21, 2021, the contents of each of which are incorporated herein by reference in their entireties.

### SEQUENCE LISTING

The instant application contains a Sequence Listing which has been submitted electronically in XML file format and is hereby incorporated by reference in its entirety. Said XML copy, created on Aug. 22, 2023, is named 4690_0036C_SL.xml and is 345,420 bytes in size.

### FIELD OF THE INVENTION

Compositions and methods are provided for delivering siRNA agents that target hepatitis B virus (HBV). The siRNA agents may be single or multiple nucleic acids and may contain nucleoside or nucleotide analogues. The siRNA agents are covalently conjugated to a peptide docking vehicle (PDoV), and are further covalently linked to one or more GalNAc ligands. Methods for treating chronic HBV infection and related diseases are provided.

### BACKGROUND OF THE INVENTION

### Hepatitis B Virus

Hepatitis B virus (HBV) is one of the most thoroughly characterized and complex hepatitis viruses. The infective particle consists of a viral core plus an outer surface coat. The core contains circular partially double-stranded DNA and DNA polymerase, and it replicates within the nuclei of infected hepatocytes. Hepatitis B is a potentially life-threatening liver infection caused by HBV. It can cause both acute and chronic infections and puts people at high risk of death from cirrhosis and liver cancer. Leung, Med. J. Malaysia; 60 Suppl B:63-6.(2005). The World Health Organization (WHO), estimated that 257 million people were living with chronic hepatitis B infection worldwide in 2015, and this number is continuous to rise despite the availability of both a preventive vaccine and of effective and well-tolerated viral suppressive medications. However, there are currently no effective treatments that can clear chronic hepatitis B infections.

More than 90% of infants that are infected will develop a chronic hepatitis B infection. Patients may be asymptomatic or have nonspecific manifestations such as fatigue and malaise. Without treatment, chronic hepatitis B can resolve (uncommon), progress rapidly, or progress slowly to cirrhosis over decades. Resolution often begins with a transient increase in disease severity and results in seroconversion from hepatitis B e antigen (HBeAg) to antibody to hepatitis B e antigen (anti-HBe). Coinfection with hepatitis D virus (HDV) causes the most severe form of chronic HBV infection; without treatment, cirrhosis develops in up to 70% of patients. Chronic HBV infection increases the risk of hepatocellular carcinoma. Christopher et al., Clin Liver Dis;. 23(3):557-572 (2019).

An acute hepatitis B infection may last up to six months (with or without symptoms) and infected persons are able to pass the virus to others during this time. A simple blood test can detect HBV in blood. Symptoms of an acute infection may include loss of appetite, joint and muscle pain, low-grade fever, and possible stomach pain. Although most people do not experience symptoms, they can appear 60-150 days after infection, with the average being 90 days or 3 months. Some people may experience more severe symptoms such as nausea, vomiting, jaundice (yellowing of the eyes and skin), or a bloated stomach that may cause them to see a health care provider.

### Current State of Anti-HBV Drugs

Immunoregulator drugs have already been used in treating severe pneumonia, immunodeficiency, and chronic hepatitis B. Jiang, Vaccine;. 30(4):758-766(2012). These drugs can improve the patients' immune response, especially the specific immunity to HBV and may help the immune cells to recognize and destroy HBV-infected cells, resulting in clearance of HBV in those destructed cells. Interferon (IFN) is a secretory glycoprotein and functions as an antiviral, anti-proliferation, and immune regulatory cytokine. Moreover, thymosin-al is another type of immunoregulators drug, and its main functions are to promote differentiation of T cells to a mature stage and enhance the response to antigens and other excitants. In addition, cytokines are synthesized and secreted by multiple types of immune cells (such as monocytes, macrophages, T cells, B cells, and NK cells) and non-immune cells (such as endothelial cells, epidermal cells, and fibroblasts) upon stimulation.

Nucleoside and nucleotide analogue drugs (collectively: NAs) have been also used in treating HBV infection. Lamivudine, a pyrimidine nucleoside drug, was the first NA approved for treating chronic HBV infection. Telbivudine is a specific, selective and oral drug used for treating chronic hepatitis B. Other drugs like Entecavir, Adefovir and Tenofovir are also oral antiviral drugs that offer potent selective inhibition of HBV infection. These NAs are incorporated into the viral DNA chain and stop the elongation of viral DNA synthesis. Kang et al., Viruses; 7:4960-77 (2015).

Each of the currently approved treatments for clinical interventions for chronic hepatitis B (CHB) require long-term therapy for most patients. Although sustained HBV DNA suppression using current therapies is associated with improvement in clinical outcomes, an excess risk of hepatocellular carcinoma (HCC) remains, and hepatitis B surface antigen (HBsAg) seroclearance is uncommon.

siRNA Therapeutics

Double-stranded RNA has been shown to silence gene expression via RNA interference (RNAi). Short-interfering RNA (siRNA)-induced RNAi regulation shows great potential to treat a wide variety of human diseases from cancer to other traditional undruggable disease, but problems remain with delivery of the siRNA to the desired tissue. In particular, improved targeting of nucleic acid drugs to specific cell types or tissues is needed, together with development of non-toxic endosomal escape agents, as explained further below.

Currently, two types of effective delivery methods for nucleic acid drugs have been used. One method uses lipid-based nanoparticle (liposomes), that contain multiple components. The other method targets the asialoglycoproteins receptor (“ASGPR”) using conjugates that contain the GalNAc molecule.

A major challenge for RNA-based therapeutics is that all pathways for delivery to cells eventually lead to endosomal escape. ASO and siRNA delivery to the liver can be achieved using ASGPR-targeted GalNAc-siRNA conjugates due to the properties of ASGPR that are well suited for macromolecular drug deliver to hepatocytes. In particular, hepatocytes express millions of copies of ASGPR on their cell surface, which cycle at a rapid rate of every 10-15 min. These properties make a GalNAc-based delivery approach effective even with a presumed endosomal escape rate of <0.01%. By contrast, effective delivery of ASO or RNA to other tissues has not been achieved. No other ligand—receptor system expresses receptors at such a high level as ASGPR,nor cycles into endosomes as rapidly. Indeed, most cell surface receptors are expressed in the range of 10,000-100,000 per cell (or lower), and caveolin and clathrin-mediated endocytosis typically recycles every 90 min. See Juliano, Nucleic Acids Res. 44, 6518-6548 (2016).

Endosomal escape remains a problem that applies to all RNA-based therapeutics. Enhancing endosomal escape by developing new chemistries and materials is needed to target the cell or tissue beyond the liver hepatocytes Small-molecule endosomolytic agents such as chloroquine have been used to disrupt or lyse endosomes, but at the effective concentration these agents invariably lyse all types of endosomes inside the cell resulting in substantial toxicity.

An alternative endosomal escape approach is to conjugate endosomolytic peptides or molecules directly to the RNA, which will strictly limit their action to endosomes containing the RNA therapeutic. Various clinical trials using a two-molecule dynamic polyconjugate (DPC) system containing cholesterol or lytic melittin peptide to escape the endosome were terminated due to toxicity effects. Wooddell, et al., Mol. Ther. 21, 973-985 (2013); Hou et al., Biotechnol. Adv. 33, 931-940 (2015).

siRNA Technology in Treating HBV Infection

RNA interference can be induced for inhibiting Hepatitis B virus replication in mammalian liver cells by siRNA molecules in a mouse model. Lian et al., J Pharmacol Sci; 114(2): 147-57 (2010). Chemically synthesized siRNA/shRNA has been demonstrated to be a possible therapeutic tool for treating hepatitis B infection. Wu et al., Virus Research, 112:100-107 (2005). However, a phase 2 trial of a drug based on RNA interference failed to show a reduction of viral burden in certain subsets of patients, where it was shown that viral antigen was being produced from integrated HBV transcripts that did not harbor the target sequence. Wooddell, supra.

Cellular factors may also contribute to RNAi resistance. Dowdy, Nature Biotechnology, 35:222-229(2017). This presents a challenge for systemic delivery of synthetic siRNA and shRNA expression vectors to specific target cells or tissues in a clinically relevant manner This challenge is also due to the fact that RNAi molecules are small, double-stranded oligonucleotides with a highly negatively charged, hydrophilic phosphate backbone. This makes them unable to interact with and cross cell membranes, and subjects them to rapid filtration from the bloodstream by the kidneys. RNAi molecules that do contact the target cells are taken up via endocytosis and remain trapped in the endosome where they are degraded by nucleolytic enzymes. Methods are needed to allow escape of intact RNAi molecules from the endosome to the cytoplasm where the RNA-induced silencing complex (“RISC”) resides.

### BRIEF DESCRIPTION OF THE FIGURES

FIG. 1 shows a graphical representation of the design of a [GalNAc] Peptide Docking Vehicle (PDoV): it has a (HₙKₘ)ₒXₚY_{q} (SEQ ID NO: 119) peptide backbone with multiple repeating units of histidine (H), lysine (K) and functional units X (amino acid, or functional linker), where n=1-10, m=1-10, o=1-10, p=1-5, q=1-5. HK repeating units have been shown to have good cell penetrating ability, and to facilitate endosome release, the lysine or the various functional unit X or Y will be adopted as the docking sites for the conjugation of ligands and Y will be adopted as the docking sites for the conjugation of oligonucleotide through a different covalent linkage. For example, the site \textcircled{1} will only be able to react in the presence of ligand such as GalNAc or other targeting ligands. The site \textcircled{3} can only conjugate with oligonucleotide and siRNA under the specific condition.

FIG. 2 shows a cartoon representation of the design of the [GalNAc] Peptide Docking Vehicle (G-PDoV). Trivalent GalNAc is covalently conjugated on one docking site A. Oligonucleotide or siRNA is conjugated on the other one or two docking sites B respectively.

FIG. 3 shows an example of the structure of the PDoV, containing one or two oligonucleotide sites and one ligand conjugation site. Figure discloses SEQ ID NOS: 127-131, respectively, in order of appearance.

FIG. 4 shows an example of a trivalent GalNAc targeting ligand molecule.

FIG. 5 shows an example of the construction of HBV siRNA-PDoV-ligand compound 1. Figure discloses SEQ ID NO: 135.

FIG. 6 shows an example of the construction of the dual HBV siRNA-PDoV-ligand compound 2. Figure discloses SEQ ID NO: 133.

FIG. 7 shows an example of the construction of the dual HBV siRNA-PDoV-ligand compound 3. Figure discloses SEQ ID NO: 134.

FIG. 8 shows the results of a primary screening assay in 293T cells. The bars shows the inhibition rate percentage on fluorescent protein expression. The siRNA molecules were incubated at 90 nM for 48 hours after co-transfection into 293T cells using lipofectamine 2000 (Life Technologies, Carlsbad, CA).

FIG. 9 shows the results of a primary screening assay in A549 cells. The bars shows the inhibition rate percentage on fluorescent protein expression. The siRNA molecules were incubated at 90 nM for 48 hours after co-transfection into 293T cells using lipofectamine 2000 (Life Technologies, Carlsbad, CA).

FIG. 10 shows EC₅₀ values for selected siRNA molecules in A549 cells.

FIGS. 11-16 show the results of siRNA inhibition in activated HepAD38 cells.

FIG. 11 shows the results of an initial screen 96h after transfection, where the y-axis measures % inhibition of HBsAg-2 production.

FIGS. 12-16 shows the results of a second round of screening of selected siRNAs in the HepAD38 cells 7 days are transfection.

FIG. 12 shows the effect on HBsAg production.

FIG. 13 shows the effect on HBeAg production.

FIG. 14 shows the effect on core particle production.

FIG. 15 shows the effect on supernatant HBV particle production.

FIG. 16 shows the effect on cccDNA production.

### SUMMARY OF THE INVENTION

What is provided is a pharmaceutical construct in which a Peptide Docking Vehicle (PDoV) is covalently linked to: (a) a targeting moiety; and (b) a first therapeutic nucleic acid, where the therapeutic nucleic acid inhibits replication of Hepatitis B virus (HBV). The therapeutic nucleic acid may be an siRNA molecule, and may be selected from the group of nucleic acids shown in Table 1 (SEQ ID NOs:1-104) or modified versions thereof. The modified siRNA may be, for example, selected from SEQ ID NOs. 105-118.

The construct may further contain a second therapeutic nucleic acid (e.g. siRNA molecule) that is the same or different from the first therapeutic nucleic acid (e.g. siRNA molecule.

The PDoV may contain multiple repeating units of histidine and lysine. Examples of PDoVs are those with structure I or II, where A and B are independently a peptide sequence of H, K, R, HH, HHH, HHHH (SEQ ID NO: 120), HHK, HHHK (SEQ ID NO: 121) or any other endosomal releasing short peptide, D is an siRNA, R_{L} is a targeting ligand, and R_{S} is a covalent linker to the therapeutic nucleic acid:

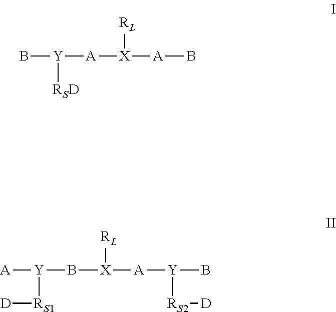

The Type X sites are used to conjugate the targeting ligands, and the Type Y sites are used to conjugate the oligonucleotide. They can be the same or different.

In some embodiments the PDoV peptide construct has a structure selected from the group consisting of PDoV 1-5 (SEQ ID NOS 127-131, respectively, in order of appearance):

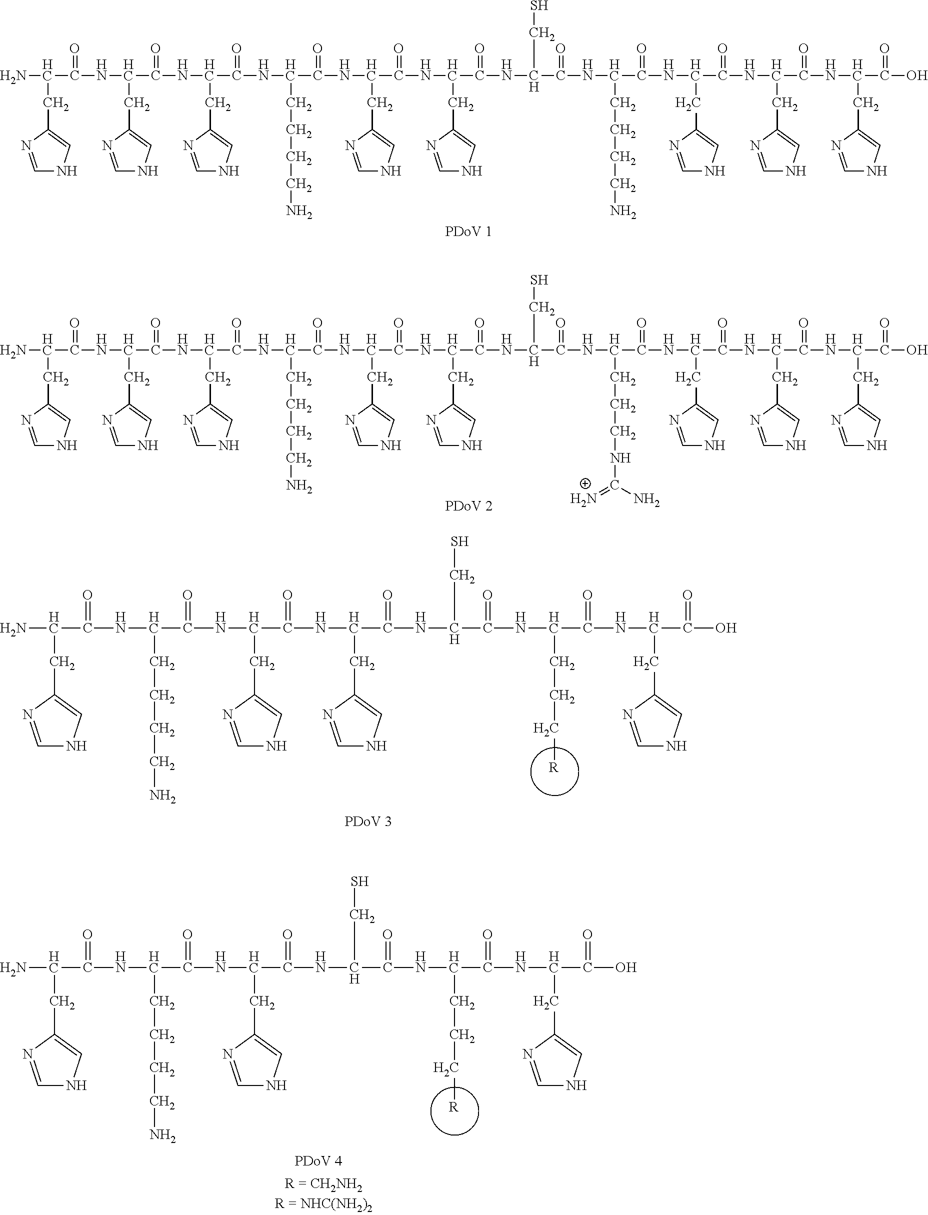

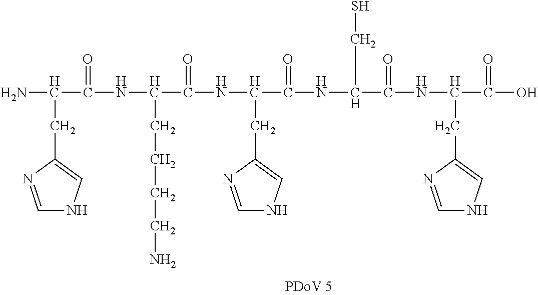 [SMILES: NCCCCC(NC(=O)C(N)Cc1c[nH]cn1)C(=O)NC(Cc1c[nH]cn1)C(=O)NC(CS)C(=O)NC(Cc1c[nH]cn1)C(=O)O]

In any of these constructs the targeting moiety may contain a ligand covalently linked to the PDoV via a linker of formula III or IV, where n is 1-3:

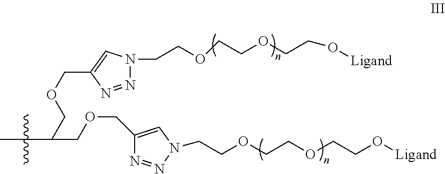

where n is 1, 2, or 3 and is connected to the dipodal linkage through a 1, 5-triazol ring with a CH₂OCH₂ unit; or

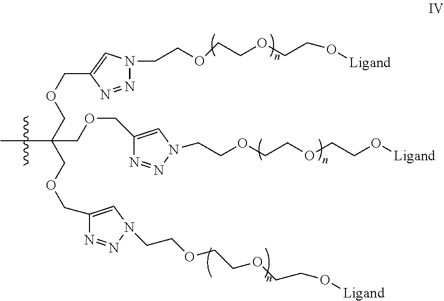

where n is 1, 2, or 3 and is connected to the tripodal linkage through a 1, 5-triazol ring with a CH₂OCH₂ unit.

In other embodiments the linker between the targeting ligand and the PdoV peptide may contain a polyethylene glycol chain —(CH₂CH₂O)ₙ-, or an alkylene chain —(CH₂CH₂)ₙ-, where n is an integer from 2-15.

In the constructs above R_{S} may be an bioorthogonal reactive moiety used to conjugate the nucleic acid with the PdoV peptide, where the reactive moiety may be, for example, an amine, hydrazine, N-hydroxysuccinimide, azido, alkyne, carboxylic acid, thiol, maleimide, phosphine diester, or a chemical reactive moiety such as:

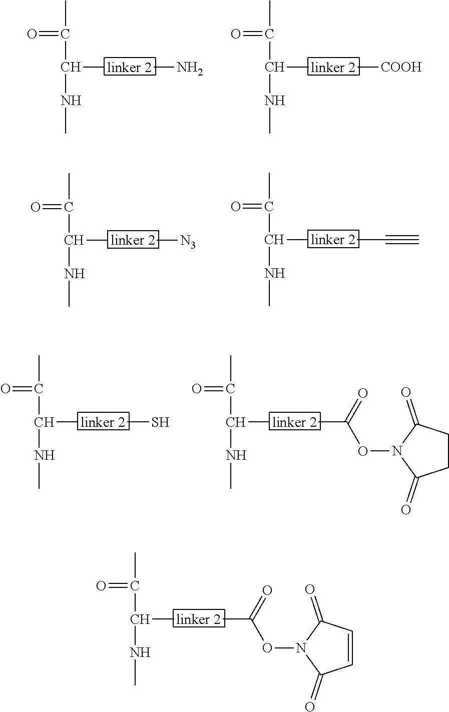

In some embodiments the siRNA molecule may contain a duplex of two complimentary, single-stranded oligonucleotides each with a length of 10-29 bases, or 19-27 bases. The construct may contain nucleic acid molecules containing deoxyribonucleotides and/or ribonucleotides.

In any of these constructs the siRNA molecule may contain at least one nucleotide chemically modified at the 2′ position. The chemically modified nucleotide may be, for example, 2′-O-methyl, 2′ -fluoro, 2′-O-methoxyethyl or 2′-O-allyl:

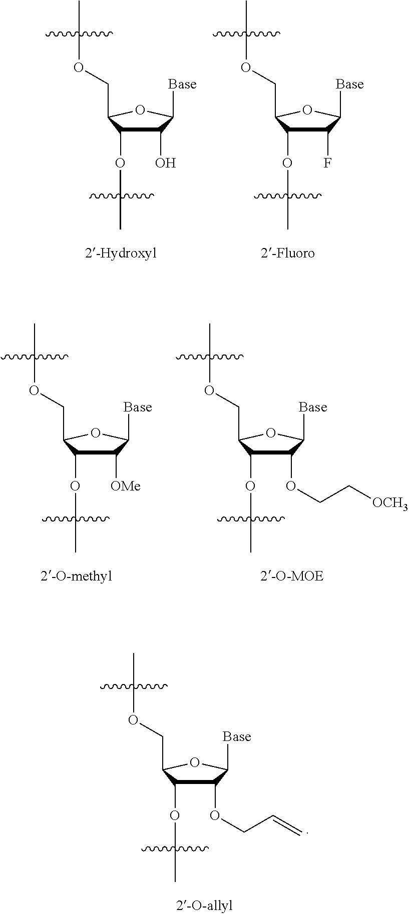

The construct may contain one or more chemically modified nucleotides selected from the group consisting of a phosphorothioate diester, phosphorodithioate diester, and a phosphoronitro diester.

In certain embodiments, the therapeutic nucleic acid is an siRNA that targets the HBV S gene, for example an siRNA with sense and antisense strands having the sequences of SEQ ID NO:38 and 38, respectively. The construct may contain a second siRNA molecule that targets, for example, the HBV S gene.

In any of these constructs the therapeutic nucleic acid may be, for example, covalently linked to the PdoV via the 5′ or 3′ position of a nucleotide or nucleoside in the nucleic acid. The linker may be an aliphatic chain, a polyethylene glycol chain, like hexanol ethylene glycol, or other hydrophobic lipid (hexanal —C₆H₁₃—) or hydrophilic chain.

In any of these constructs the targeting ligand may be, for example, N-acetyl-galactosamine (GalNAc), galactose, galactosamine, N-formal-galactosamine, N-propionyl-galactosamine, or N-butanoylgalactosamine Advantageously, the targeting ligand is N-acetyl-galactosamine (GalNAc).

In particular embodiments, the PdoV contains a C-terminal sequence having a C-terminal cysteine. The C-terminal sequence may, for example, contain the sequence [KHHHKHHHHnKHHHKHHHK]₂KXC (SEQ ID NO: 122) where n=0 or 1 and where X is a synthetic molecule linker (C₆H₁₃, —(CH₂CH₂O)ₙ- linker, n=2-12) or a peptide linker such as serine, SSS, SSSS (SEQ ID NO: 123), SSSSS (SEQ ID NO: 124), SSSSSS (SEQ ID NO: 125), or TTTT (SEQ ID NO: 126) between the terminal cysteine and a therapeutic molecule, such as a molecule used to treat HBV. The therapeutic molecule may be, for example, lamivudine, adefovir, entecavir, telbivudine, or tenofovir.

In any of these constructs the construct may have the structure (SEQ ID NOS 132-134, respectively, in order of appearance):

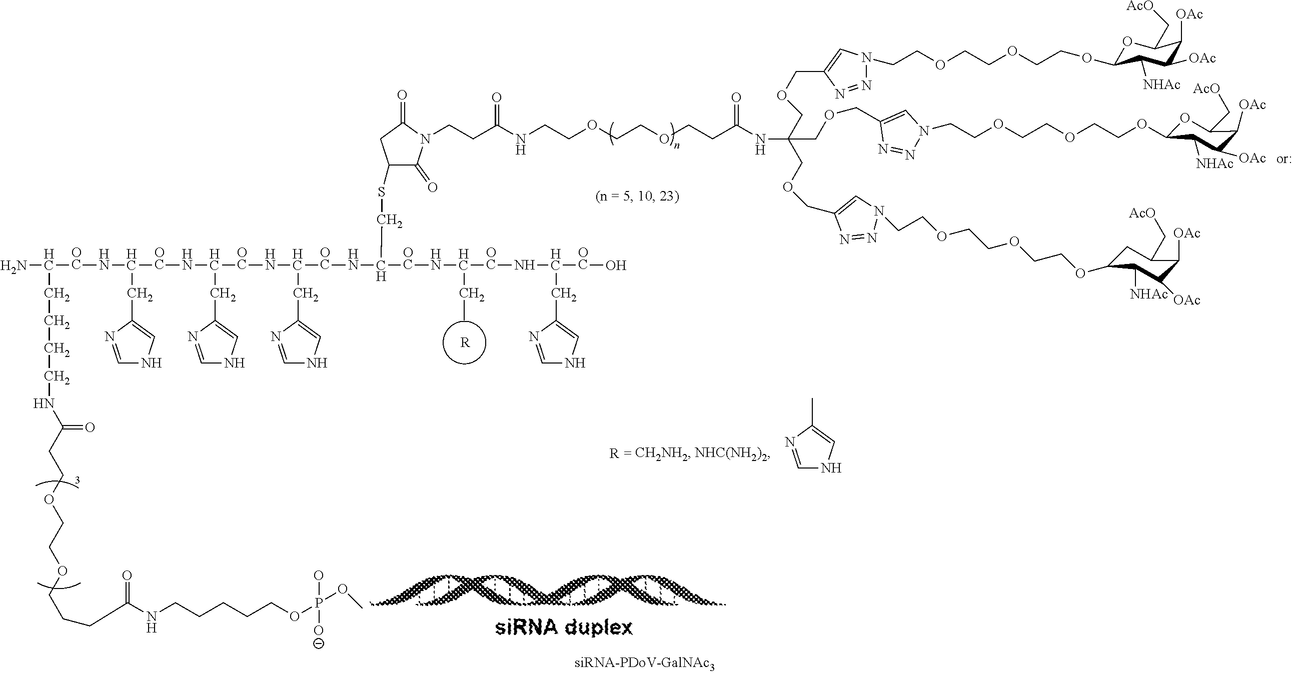

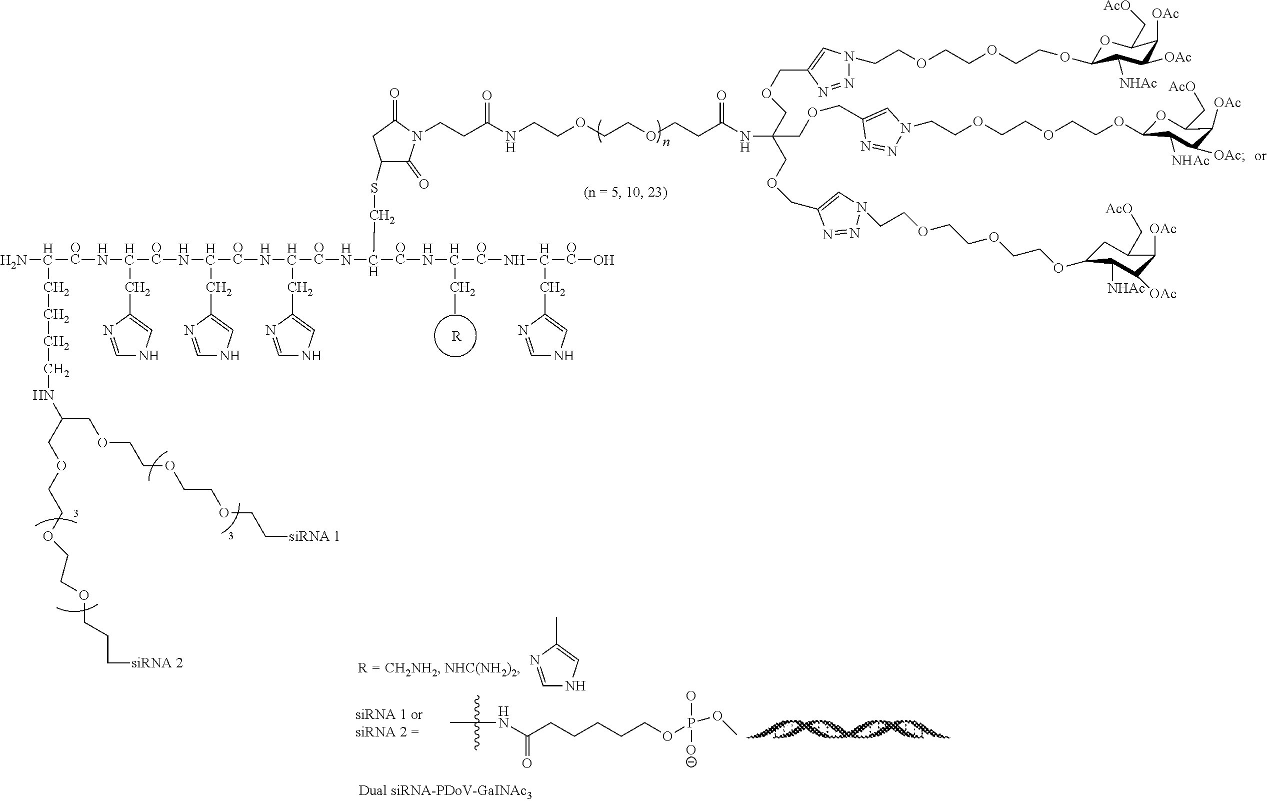

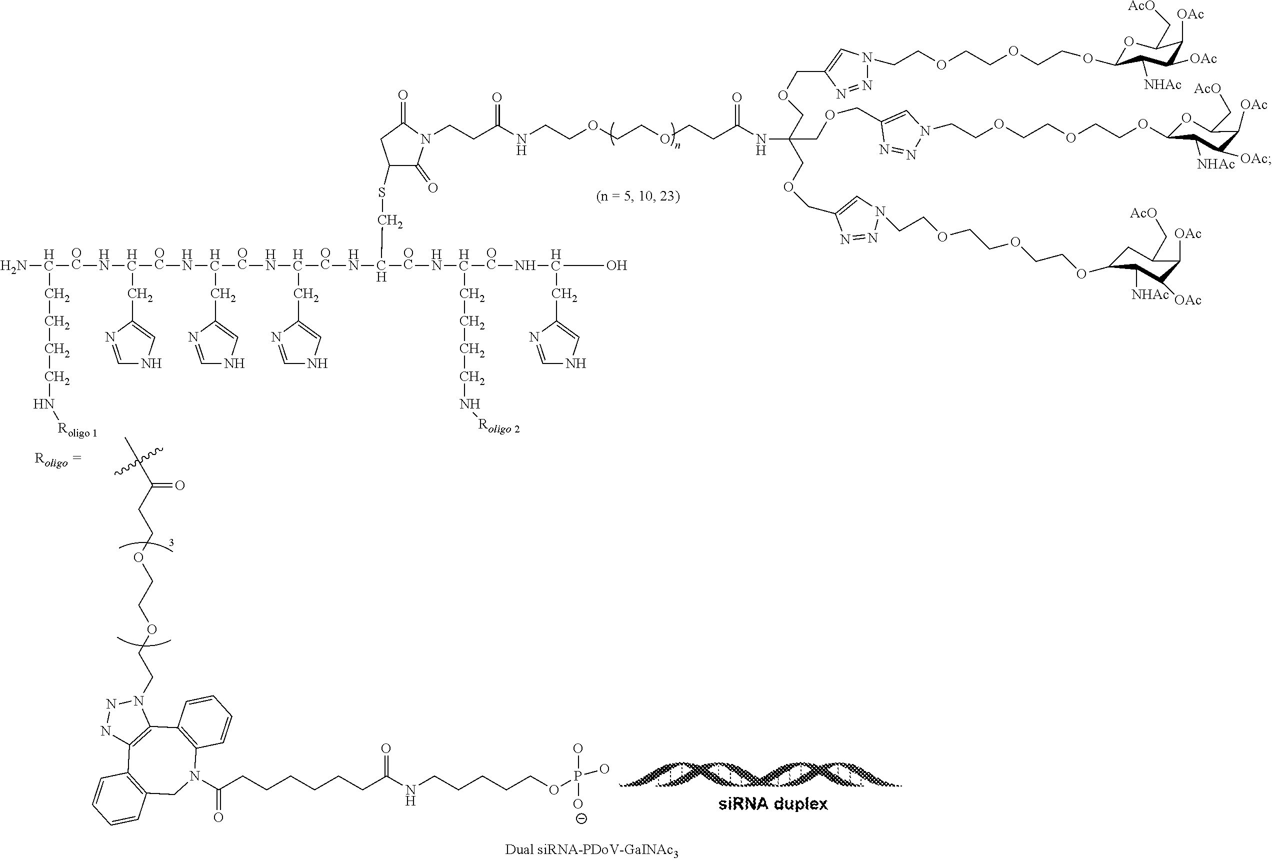

Also provided are pharmaceutical compositions containing a construct as described above and a pharmaceutically acceptable carrier. The pharmaceutically acceptable carrier may contain water and one or more salts or buffers selected from the group consisting of potassium phosphate monobasic anhydrous NF, sodium chloride USP, sodium phosphate dibasicheptahydrate USP, glucose, and Phosphate Buffered Saline (PBS).

Further provided are methods of treating HBV in a subject, in which a pharmaceutical composition as described above is administered to the subject, such as a human subject.

### DETAILED DESCRIPTION

Compositions and methods using interfering RNA molecules having enhanced therapeutic benefit are provided. The compositions and methods allow targeted cell/tissue delivery of a therapeutic compound, such as an siRNA molecule, to a subject by linking a targeting ligand to the compound. The subject may be an animal or a human.

In some embodiments, the targeting ligand as described herein may be conjugated to an endosome releasing peptide through an orthogonal bioconjugation method. The targeting ligand may particularly be used to improve the delivery of RNAi molecules to a selected target, such as the liver. In other embodiments, the targeting ligand(s) permit targeted delivery of RNAi molecules into other tissues, for example, in the skin and brain.

The targeting ligands as described herein may include one or more targeting moieties, one or more linkers. The linkers covalently conjugated with the siRNA and targeting ligands through click chemistry, thiol/maleimide chemistry, or other bioorthogonal chemistry. Linkers advantageously are hydrophilic and can be, for example, a water soluble flexible polyethylene glycol (PEG) which is sufficiently stable and limits the potential interaction between one or more targeting moiety(s). PEG has been validated to be safe and compatible for therapeutic purposes from clinical studies. In some embodiments, the linker can be poly(L-lactide)n (where n=5-20) of defined molecular weight, where the ester bond is enzymatically or hydrolytically labile.

The targeting ligand may include one or more targeting moieties, one or more groups with a linker reactive connection moiety. They are covalently conjugated with the siRNA and targeting ligands through click chemistry, thiol/maleimide chemistry, or other bioorthogonal chemistry. The linker reactive connection moiety may be, but is not limited to, a thiol-maleimide linkage, a triazol linkage formed by reaction of an alkyne and an azide, and an amide formed from an amine-NHS ester linkage. Each of these linkages is suitable for covalently linking both the targeting ligands and the therapeutic compound.

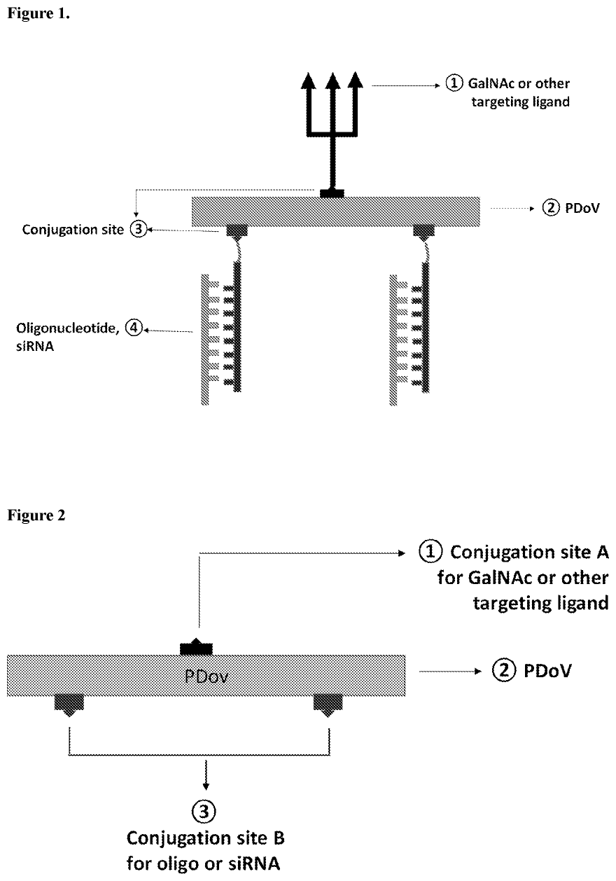

In some embodiments, the targeting specific RNA compound disclosed herein can be directly conjugated to an endosome releasing docking peptide via the 3′ or 5′ terminal end of the RNA. The targeting ligand (for example N-acetyl-galactosamine) may also be conjugated with the same docking peptide in a compatible method. FIG. 1 shows a cartoon representation of the design of an exemplary [GalNAc] Peptide Docking Vehicle (G-PDoV). Trivalent GalNAc is covalently conjugated on one docking site A. Oligonucleotide or siRNA is conjugated on the other one or two docking sites B respectively.

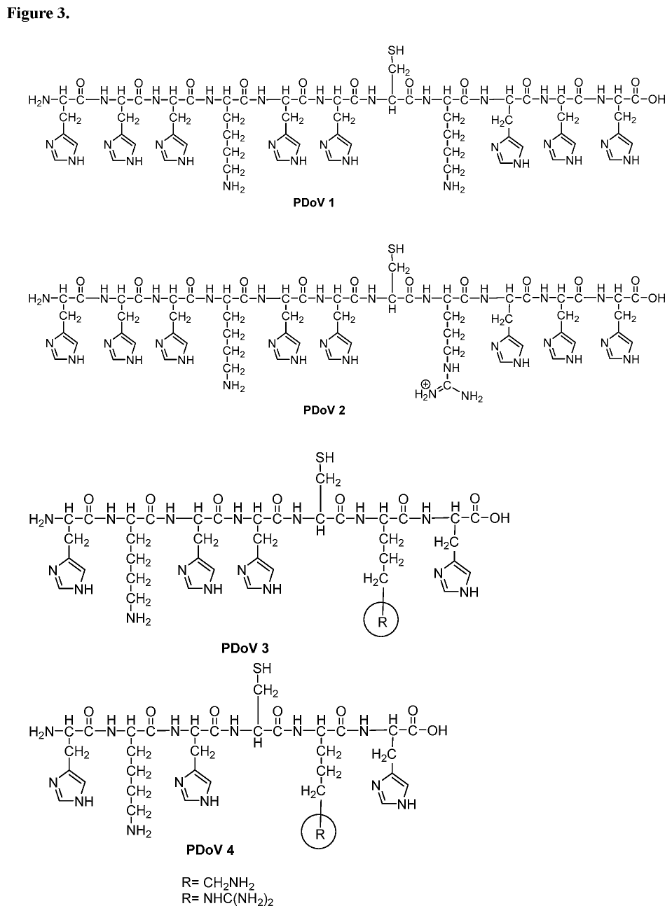

FIG. 2 shows a graphical representation of the design of a [GalNAc] Peptide Docking Vehicle (PDoV): it has a (HₙKₘ)ₒXₚY_{q} (SEQ ID NO: 119) peptide backbone with multiple repeating units of histidine (H), lysine (K) and functional units X (amino acid, or functional linker), where n=1-10, m=1-10, o=1-10, p=1-5, q=1-5. HK repeating units have been shown to have good cell penetrating ability, and to facilitate endosome release, the lysine or the various functional unit X or Y will be adopted as the docking sites for the conjugation of ligands and Y will be adopted as the docking sites for the conjugation of oligonucleotide through a different covalent linkage. For example, the site \textcircled{1} will only be able to react in the presence of ligand such as GalNAc or other targeting ligands. The site \textcircled{3} can only conjugate with oligonucleotide and siRNA under the specific condition. FIG. 3 shows examples of some PDoV peptides. In each peptide differential coupling can be achieved via the thiol and amino groups, which allow selective conjugation of moieties containing targeting moieties and therapeutic nucleic acids. Additional conjugation schemes are described in WO/2021/113851.

In some embodiments, the targeting specific RNA compound disclosed herein can also be directly conjugated to a targeting ligand (for example N-acetyl-galactosamine), via, for example, the 3′ or 5′ terminal end of the RNA. In some embodiments, the RNA may contain one or more modified nucleotides such as 3′-OMe, 3′-F, or 3′-MOE. In some embodiments, the RNA can be an RNAi agent, for example a double stranded RNAi agent. In some embodiments, the targeting ligands disclosed herein are linked to the 5′ or 3′ terminus of the sense strand of a double stranded RNAi agent or the 5′ or 3′ terminus of the antisense strand of a double stranded RNAi agent. The targeting ligands may alternatively be linked to both 3′/3′, 3′/5′ or 5′/5′ terminal end of the sense and antisense strand of a double stranded RNAi agent. The targeting ligands may be covalently bonded to the RNAi molecule via, for example, a phosphate, phosphorothioate, or phosphonate group at the 3′ or 5′ terminus of the sense strand of a double stranded RNAi agent.

In some embodiments, the targeting specific RNA molecule disclosed herein is an siRNA molecule targeted to a hepatitis B Virus (HBV) mRNA and that inhibits expression of that HBV mRNA. As used herein, the term “target” advantageously refers to a sequence of one or more nucleotides in a gene of HBV.

In some embodiments, the target sequence for a selected siRNA is in the Surface gene (HBsAg) in the HBV genome. This region is specific for HBV and is highly conserved. The main function of the HBV surface antigen proteins is to form the HBV envelope.

In some embodiments, the target sequence for a selected siRNA is in the core protein (HBcAg) gene in the HBV genome. HBcAg forms dimers and has arginine-rich motifs at the C-terminus that are believed to interact with the viral nucleic acid in the nucleocapsid.

In some embodiments, the target sequence for a selected siRNA is in the E antigen (HBeAg) gene in the HBV genome. The HBeAg ORF encodes an endoplasmic reticulum (ER) targeting sequence that co-translationally traffics the peptide to the ER, where the protein is processed to the final 15 kD HBeAg that is secreted from HBV-infected cells.

In some embodiments, the target sequence for a selected siRNA is in the X (HBx) gene in the HBV genome.HBx is the only regulatory protein encoded by HBV, which plays an essential role during HBV replication. Specifically, studies have shown that HBx is bound to cccDNA, that HBx is required for transcription from cDNA, and that downstream HBx-mediated effects are required for HBV replication.

In some embodiments, the target sequence for a selected siRNA is in the polymerase (HBp)/reverse transcriptase (RT) genes in the HBV genome. The polymerase plays a critical role during the HBV life cycle, and these two enzyme activities are critical for HBV replication during the life cycle of HBV proliferation.

In some embodiments, an HBV's gene-targeted siRNA comprises a sense strand and an antisense strand, each containing a core sequence that is 19-21 nucleobases in length. The length of the herein described siRNA sense and antisense strands are independently 19 to 27 nucleotides in length.

In some embodiments, the length of the siRNA sense and antisense strands are independently 21 or 25 nucleotides in length. The sense and antisense strands of siRNA typically anneal to form a duplex. Within the complementary duplex region, the sense strand core sequence is 100% complementary to the antisense core sequence.

In some embodiments, the siRNAs may have an asymmetric structure, where the sense strand is about 19 nucleotides in length while the antisense strand is about 21 nucleotides in length.

These and other aspects of the invention are described in more detail below.

### Definitions:

As used herein, “oligonucleotide” refers to a chemically modified or unmodified nucleic acid molecule (RNA or DNA) having a length of less than 100 nucleotides (for example less than 50, less than 30, or less than 25 nucleotides). It can be siRNA, microRNA, anti microRNA, microRNA mimics, dsRNA, ssRNA, aptamer, triplex forming oligonucleotides, aptamers. In one embodiment, the oligonucleotide is an RNAi agent.

As used herein, an “siRNA molecule” or “RNAi molecule” is a duplex oligonucleotide, that is a short, double-stranded polynucleotide, that interferes with the expression of a gene in a cell, after the molecule is introduced into the cell. For example, an siRNA molecule targets and binds to a complementary nucleotide sequence in a single stranded target RNA molecule. By convention, when an siRNA molecule is identified by a particular nucleotide sequence, the sequence refers to the sense strand of the duplex molecule. One or more of the ribonucleotides comprising the molecule can be chemically modified by techniques known in the art. In addition to being modified at the level of one or more of its individual nucleotides, the backbone of the oligonucleotide can be modified. Additional modifications include the use of small molecules (e.g. sugar molecules), amino acids, peptides, cholesterol, and other large molecules for conjugation onto the siRNA molecule.

“Peptide Docking Vehicle” (PDoV) refers to a synthetic peptide of defined sequence that contains multiple conjugation sites to allow conjugation with one or more targeting ligands and with one or more oligonucleotides. It contains functional groups, such as a hydrophobic chain or a pH sensitive residue, which facilitate the release of the oligonucleotide payload entrapped inside of the endosome of a cell after delivery of the conjugated PDoV to the cell. Suitable docking vehicles for use in the embodiments described herein are detailed in WO/2021/113851, the contents of which are hereby incorporated by reference in their entirety.

“Inhibition of expression” refers to the absence or significantly decreasing in the level of protein and/or mRNA product from the target gene. The inhibition does not have to be absolute, but may be partial inhibition sufficient for there to a detectable or observable change as a result of the administration of a siRNA molecule of the invention. Inhibition may be measured by determining a decrease in the level of mRNA and/or protein product from a target nucleic acid relative to a cell lacking the siRNA molecule, and maybe as little as 10%, 50%, or maybe absolute i.e. 100% inhibition. The effects of inhibition may be determined by examination of the outward properties i.e. quantitative and/or qualitative phenotype of the cell or organism, and may also include an assessment of the viral load following administration of a siRNA molecule of the invention.

siRNA molecules can directly target the activity of genes with minimum off-target events. By “off target events” it is meant that expression of nucleic acids are not inhibited by the siRNA molecules other than the target are decreased significantly. In the case of HBV infection, this offers a unique opportunity to address the unmet clinical treatment needs for HBV. Accordingly, in one aspect of the invention, there is provided HBV DNA-directed RNA interference agents for inhibiting expression of one or more target sequences in an HBV gene.

### Structure of the PDoV

An endosomal escape peptide (PDoV)is provided that enhances escape of macromolecular cargo, such as an siRNA molecule, into the cytoplasm in a non-toxic manner A schematic diagram of an exemplary PDoV molecule is shown in FIG. 2. Various examples of the PDoV platform are shown in FIG. 3. In the PDoV the endosomal escaping peptide acts both as the docking site linker for the RNA and the targeting ligands. Multiple RNA molecules can be conjugated with the same construct to achieve codelivery of siRNA molecules against different target mRNAs, thereby providing a synergistic benefit for silencing a multi-disease related gene. The histidine and lysine rich polypeptide or linear histidine and lysine rich peptide has been shown to be an effective cell penetrating and endosomal release agent in the delivery of RNA. The peptide contains a histidine rich domain, where the imidazole rings of the histidine residues are protonated at a lower pH value (pH<˜6) and act inside the endosome as a proton sponge, which leads to lysis of the endosome lipid bilayers and release of the RNA. The conjugation sites on the PDoV are described in more detail below.

FIG. 2 shows a graphical representation of the design of a [GalNAc] Peptide Docking Vehicle. A trivalent GalNAc ligand is covalently conjugated on one docking site 1 of the peptide, while an oligonucleotide or siRNA is conjugated on the other one or two docking sites 3 respectively.

The PDoV advantageously contains multiple repeating units of histidine and lysine and may have the structure, for example, I or II:

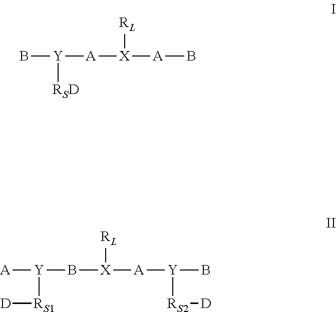

where A and B are independently a peptide sequence of H, K, R, HH, HHH, HHHH (SEQ ID NO: 120), HHK, HHHK (SEQ ID NO: 121) or any other endosomal releasing short peptide, D is an siRNA, R_{L} is a targeting ligand, and R_{S} is a covalent linker to the nucleic acid. The Type X sites are used to conjugate the targeting ligands, and the Type Y sites are used to conjugate the oligonucleotide. They can be the same or different. Typical X and Y sites contain reactive amino and/or sulfhydryl moieties.

Examples of suitable PDoV peptides are shown below (SEQ ID NOS 127-131, respectively, in order of appearance):

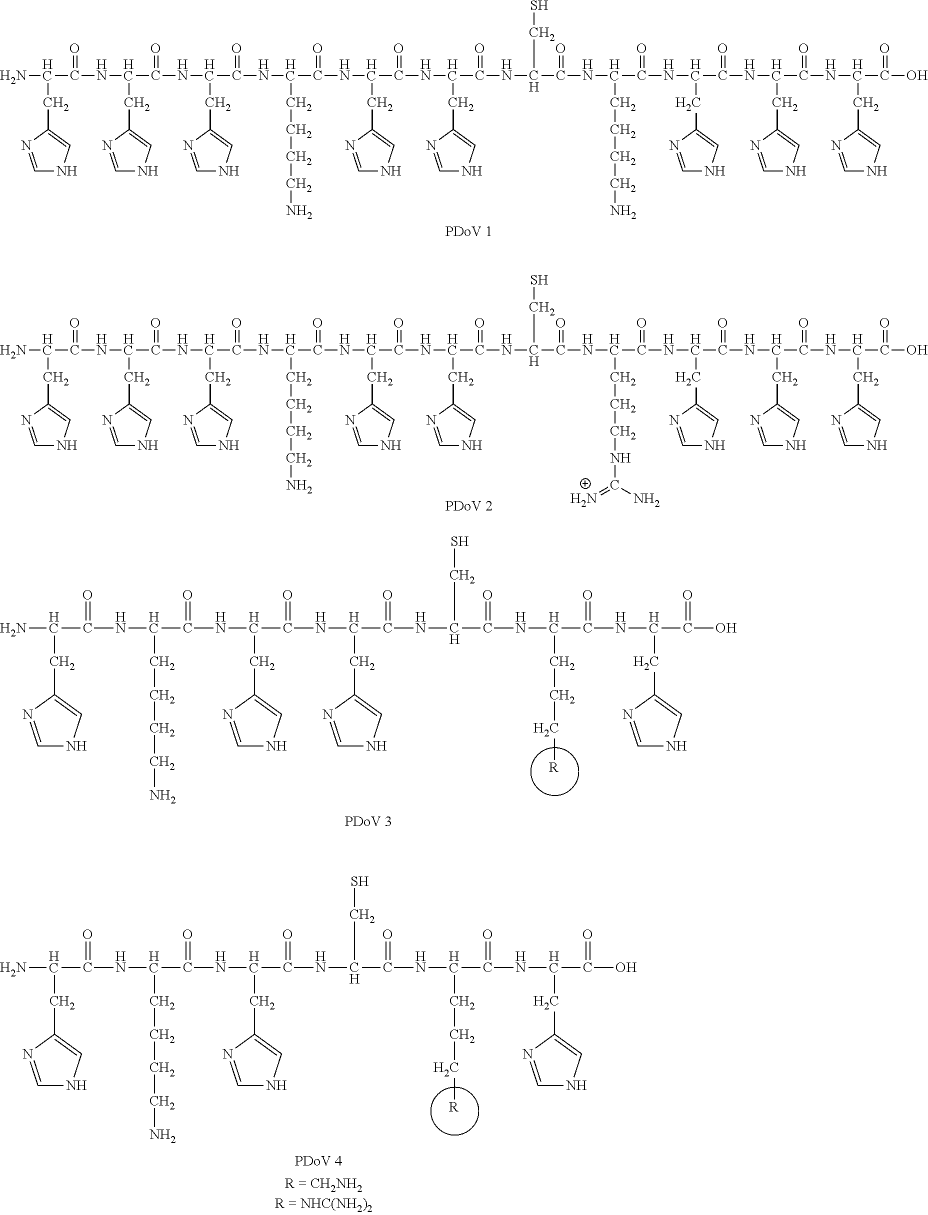

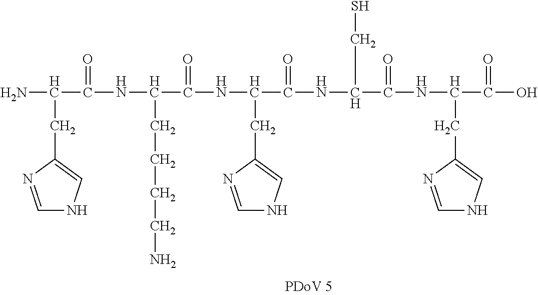 [SMILES: NCCCCC(NC(=O)C(N)Cc1c[nH]cn1)C(=O)NC(Cc1c[nH]cn1)C(=O)NC(CS)C(=O)NC(Cc1c[nH]cn1)C(=O)O]

The targeting moiety in a PDoV construct contains a ligand covalently linked to the PDoV peptide via a linker of formula III or IV, wherein n is 1-3:

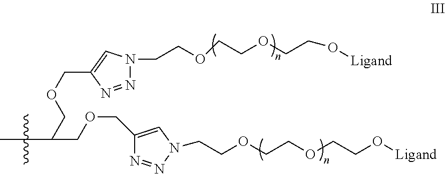

where n is 1, 2, or 3 and is connected to the dipodal linkage through a 1, 5-triazol ring with a CH₂OCH₂ unit; or

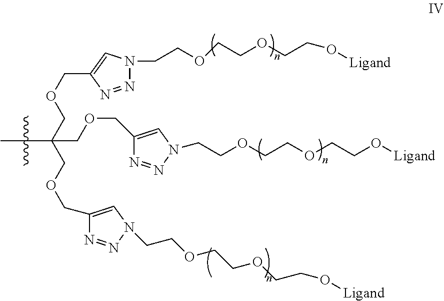

where n is 1, 2, or 3 and is connected to the tripodal linkage through a 1, 5-triazol ring with a CH₂OCH₂ unit.

The linker between the targeting ligand and the PDoV peptide may contain a polyethylene glycol chain —(CH₂CH₂O)ₙ, or an alkylene chain —(CH₂CH₂)ₙ— chain, where n is an integer from 2-15.

In structures I and II above R_{S} is a bioorthogonal reactive moiety to conjugate the nucleic acid with the PDoV peptide, whereithe reactive moiety may be, for example, an amine, hydrazine, N-hydroxysuccinimide, azido, alkyne, carboxylic acid, thiol, maleimide, phosphine diester, or a chemical reactive moiety as shown below:

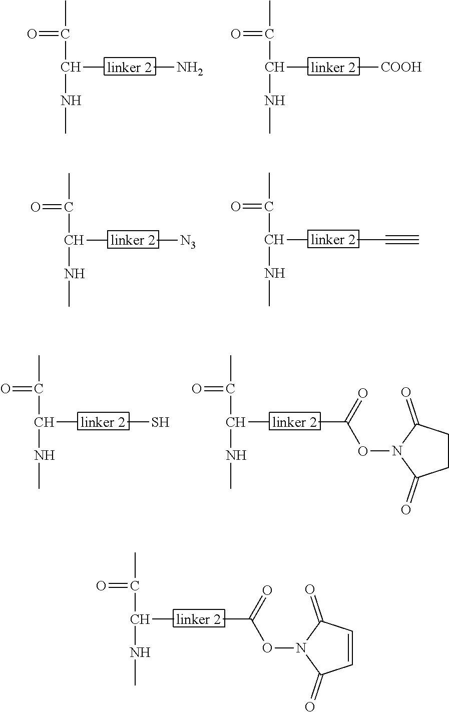

### RNAi Agents

HBV has a small circular DNA genome, ≈3.2 kb in length, that contains 4 genes with partially overlapping open reading frames (ORF_{S}). These ORFs encode the polymerase protein (P gene); core antigen and e antigen (C gene); large, medium, and small surface-antigen proteins (S gene); and the X protein (X gene). The hepatitis B virus (HBV) genome exists in two forms: circular covalently closed DNA (cccDNA) and relaxed circular DNA (rcDNA). The gene organization of HBV DNA is highly conserved between all viral subtypes, which is useful for gene silencing.

siRNA molecules were designed and selected to target the sequences of at least seven of HBV strains to cover as many mutants of HBV as possible. Two lengths for the siRNA molecules were used: 21-mer (19+dTdT) and 25-mer.

The double strand siRNA may be unmodified or chemically modified using modification that are well known in the art. For example, one or more of the RNA nucleotides may be modified at the 2′ position with 2′-F or 2′-OMe, and/or at the 5′-position with —P(O)₂=S, —P(S)₂=O. Other chemical modifications, such as replacement of the phosphodiester linkage with a linkage resistant to RNAse activity, such as a phosphorothioate may be used. The siRNA may also be modified by pegylation or lipid functionalization to improve the overall stability and bioavailability of the RNAi.

In one embodiment, the siRNA molecule or other nucleic acid has a length of 19 to 27 base pairs of nucleotides; in another embodiment, the siRNA molecule or other nucleic acid has a length of 20 to 30 base pairs; in still another embodiment the siRNA molecule or other nucleic acid has a length of 24 to 28 base pairs. The molecule can have blunt ends at both ends, or sticky ends at both ends, or one of each. The siRNA molecule may include a chemical modification at the individual nucleotide level or at the oligonucleotide backbone level, or it may have no modifications. In one preferred embodiment an anti-HBV siRNA possesses strand lengths of 25 nucleotides. In another, an anti-HBV siRNA possesses strand lengths of 19 to 25 nucleotides. In some embodiments, the siRNA molecules can be asymmetric where one strand is shorter than the other (typically by 2 bases e.g. a 21mer with a 23mer or a 19mer with a 21mer or a 23mer with a 25mer). The strands may be modified by inclusion of a dTdT overhang group on the 3′ end of selected strands, where the dTdT overhang is not counted in the strand length as described above.

### Modifications and Linkages

A dsRNA agent of the disclosed embodiments can be conjugated (e.g., at its 5′ or 3′ terminus of its sense or antisense strand) or unconjugated to another moiety (e.g., a non-nucleic acid moiety such as a peptide), an organic compound (e.g., a dye, cholesterol, or the like). Modifying dsRNA agents in this way may improve cellular uptake or enhance cellular targeting activities of the resulting dsRNA agent derivative as compared to the corresponding unconjugated dsRNA agent, are useful for tracing the dsRNA agent derivative in the cell or improve the stability of the dsRNA agent derivative compared to the corresponding unconjugated dsRNA agent.

As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphorodithioates, phosphoramidates, methyl phosphonates, chiral-methyl phosphonates, 2′-O-methyl ribonucleotides, 2′-Fluoro ribonucleotides, peptide-nucleic acids (PNAs) and unlocked nucleic acids (UNAs; see, e.g., Jensen et al. Nucleic Acids Symposium Series 52: 133-4), and derivatives thereof.

As used herein, “nucleotide” is used as recognized in the art to include those with natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other, see, e.g., Usman and McSwiggen, supra; Eckstein, et al., International PCT Publication No. WO 92/07065; Usman et al, International PCT Publication No. WO 93/15187; Uhlman & Peyman, supra. There are several examples of modified nucleic acid bases known in the art as summarized by Limbach, et al, Nucleic Acids Res. 22:2183, 1994. Examples of base modifications that can be introduced into nucleic acid molecules include, hypoxanthine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin, et al., Biochemistry 35:14090, 1996; Uhlman & Peyman, supra). A modified base indicates a nucleotide base other than adenine, guanine, cytosine and uracil at the 1′ position or their equivalents.

As used herein, a modified nucleotide or modified residue refers to a nucleotide having one or more modifications, typically non-naturally occurring modifications, to the nucleoside, the base, pentose ring, or phosphate group, although modifications may include naturally occurring modifications produced by enzymes that modify nucleotides, such as methyltransferases. Non-naturally occurring modifications in nucleotides include those with 2′ modifications, e.g., 2′-methoxy (2′-OMe), 2′-methoxyethoxy, 2′-fluoro (2′-F), 2′-allyl, 2′-O-[2-(methylamino)-2-oxoethyl], 4′-thio, 4′-CH2-O-2′-bridge, 4′-(CH2)2-O-2′-bridge, 2′-LNA or other bicyclic or “bridged” nucleoside analog, and 2′-O-(N-methylcarbamate) or those comprising base analogs.

As used herein, an amino modification means 2′-NH2 or 2′-O-NH2, which can be further modified, or be unmodified. Such modified groups are described, e.g., in Eckstein et al., U.S. Pat. No. 5,672,695 and Matulic-Adamic et al., U.S. Pat. No. 6,248,878. “Modified nucleotides” of the disclosed embodiments can also include nucleotide analogs as described above.

In reference to the nucleic acid molecules of the present disclosure, modifications may exist upon these agents in patterns on one or both strands of the double stranded ribonucleic acid (dsRNA). As used herein, modification at “alternating positions” indicates that every other nucleotide is a modified nucleotide or there is an unmodified nucleotide (e.g., an unmodified ribonucleotide) between every modified nucleotide over a defined length of a strand of the dsRNA (e.g., 5′-MNMNMN-3′; 3′-MNMNMN-5′; where M is a modified nucleotide and N is an unmodified nucleotide). The modification pattern starts from the first nucleotide position at either the 5′ or 3′ terminus according to a position numbering convention. The pattern of modified nucleotides at alternating positions may run the full length of the strand, but in certain embodiments includes at least 4, 6, 8, 10, 12, 14 nucleotides containing at least 2, 3, 4, 5, 6 or 7 modified nucleotides, respectively. Modifications with alternating pairs of positions indicates a pattern where two consecutive modified nucleotides are separated by two consecutive unmodified nucleotides over a defined length of a strand of the dsRNA (e.g., 5′-MMNNMMNNMMNN-3′; 3′-MMNNMMNNMMNN-5′; where M is a modified nucleotide and N is an unmodified nucleotide). The modification pattern starts from the first nucleotide position at either the 5′ or 3′ terminus according to a position numbering convention such as those described herein. The pattern of modified nucleotides at alternating positions may run the full length of the strand, but preferably includes at least 8, 12, 16, 20, 24, 28 nucleotides containing at least 4, 6, 8, 10, 12 or 14 modified nucleotides, respectively. These modification patterns are exemplary and the skilled artisan will recognize that additional patterns may be used.

In certain embodiments, the first and second oligonucleotide sequences of the siRNA exist on separate oligonucleotide strands that can be and typically are chemically synthesized. In some embodiments, both strands contain 19 nucleotides, or both strands contain 25 nucleotides. These molecules may completely complementary and have blunt ends, or may have dTdT overhangs on one or both strands. In certain embodiments the siRNA strands have differing lengths, with one possessing a blunt end at the 3′ terminus of a first strand (sense strand) and a 3′ overhang at the 3′ terminus of a second strand (antisense strand). The siRNA can also contain one or more deoxyribonucleic acid (DNA) base substitutions.

Suitable siRNA compositions that contain two separate oligonucleotides can be chemically linked outside their annealing region by chemical linking groups. Many suitable chemical linking groups are known in the art. Suitable groups will not block endonuclease activity on the siRNA and will not interfere with the directed destruction of the RNA transcribed from the target gene. Alternatively, the two separate oligonucleotides can be linked by a third oligonucleotide such that a hairpin structure is produced upon annealing of the two oligonucleotides making up the siRNA composition. The hairpin structure will not block endonuclease activity on the siRNA and will not interfere with the directed destruction of the target RNA.

The dsRNA molecules of the disclosed embodiments are added directly, or can be complexed with lipids (e.g., cationic lipids), packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers.

The selected siRNA molecules inhibit the expression of HBV nucleic acid sequences. Advantageously, the HBV target gene is the nucleic acid sequence that is expressed as the polymerase (P) gene. Accordingly, in one embodiment, the siRNA agents inhibit expression of one or more target sequences in the HBV P gene. The HBV genome has overlapping open reading frames. As such, targeting particular sequences of the polymerase gene will also target the same sequences in the overlapping gene. The siRNA molecules of the invention therefore are capable of targeting multiple genes with a single effector sequence. In each embodiment described below, however, at least the polymerase gene is targeted. Because the genes of the HBV genome, in most regions, overlap among the four ORFs, the siRNA molecules of the invention are also capable of targeting the C gene, S gene, and X gene of the HBV. Based on the sequences conserved between eleven HBV strains, 52 siRNAs were designed as shown in Table 1.

TABLE 1
siRNA sequence targeting HBV conserved genes:

| | | SEQ | | SEQ |
|---|---|---|---|---|
| NTS# | | ID | | ID |
| SARNA | SS (5′-3′) | NO: | AS (5′-3′) | NO: |
| HBV- | GGUUCUUCUGGACUAUCAAdTdT | 1 | UUGAUAGUCCAGAAGAACCdTdT | 2 |
| 21-1# | | | | |
| HBV- | CGCUUGGGACUCUCUCGUCdTdT | 3 | GACGAGAGAGUCCCAAGCGdTdT | 4 |
| 21-2# | | | | |
| HBV- | CGCAGUCCCCAACCUCCAAdTdT | 5 | UUGGAGGUUGGGGACUGCGdTdT | 6 |
| 21-3# | | | | |
| HBV- | CCAACCUCCUGUCCUCCAAdTdT | 7 | UUGGAGGACAGGAGGUUGGdTdT | 8 |
| 21-4# | | | | |
| HBV- | GUCCUGGUUAUCGCUGGAUdTdT | 9 | AUCCAGCGAUAACCAGGACdTdT | 10 |
| 21-5# | | | | |
| HBV- | CCUCUUCAUCCUGCUGCUAdTdT | 11 | UAGCAGCAGGAUGAAGAGGdTdT | 12 |
| 21-6# | | | | |
| HBV- | CUCCUGCUAUGCCUCAUCUdTdT | 13 | AGAUGAGGCAUAGCAGCAGdTdT | 14 |
| 21-7# | | | | |
| HBV- | GACGCAACCCCCACUGGCUdTdT | 15 | AGCCAGUGGGGGUUGCGUCdTdT | 16 |
| 21-84 | | | | |
| HBV- | CUCUCGUCCCCUUCUCCGUdTdT | 17 | ACGGAGAAGGGGACGAGAGdTdT | 18 |
| 21-9# | | | | |
| HBV- | GGGCUUUCCCCCACUGUUUdTdT | 19 | AAACAGUGGGGGAAAGCCCdTdT | 20 |
| 21-10# | | | | |
| HBV- | CCUAUUGAUUGGAAAGUAUdTdT | 21 | AUACUUUCCAAUCAAUAGGdTdT | 22 |
| 21-11# | | | | |
| HBV- | GCAGUCCCCAACCUCCAAUCACUCA | 23 | UGAGUGAUUGGAGGUUGGGGACUGC | 24 |
| 25-1# | | | | |
| HBV- | GUCCCCAACCUCCAAUCACUCACCA | 25 | UGGUGAGUGAUUGGAGGUUGGGGAC | 26 |
| 25-2# | | | | |
| HBV- | CCAACCUCCAAUCACUCACCAACCU | 27 | AGGUUGGUGAGUGAUUGGAGGUUGG | 28 |
| 25-3# | | | | |
| HBV- | GUCCUGGUUAUCGCUGGAUGUGUCU | 29 | AGACACAUCCAGCGAUAACCAGGAC | 30 |
| 25-4# | | | | |
| HBV- | CUCUUCAUCCUGCUGCUAUGCCUCA | 31 | UGAGGCAUAGCAGCAGGAUGAAGAG | 32 |
| 25-5# | | | | |
| HBV- | GUUGCCCGUUUGUCCUCUAAUUCCA | 33 | UGGAAUUAGAGGACAAACGGGCAAC | 34 |
| 25-6# | | | | |
| HBV- | GUGCACUUCGCUUCACCUCUGCACG | 35 | CGUGCAGAGGUGAAGCGAAGUGCAC | 36 |
| 25-7# | | | | |
| HBV- | CCUGCUGGUGGCUCCAGUUdTdT | 37 | AACUGGAGCCACCAGCAGGdTdT | 38 |
| 21-12# | | | | |
| HBV- | CAGCAAUGUCAACGACCGAdTdT | 39 | UCGGUCGUUGACAUUGCUGdTdT | 40 |
| 21-13# | | | | |
| HBV- | CCUUGGACUCAUAAGGUGGdTdT | 41 | CCACCUUAUGAGUCCAAGGdTdT | 42 |
| 21-14# | | | | |
| HBV- | GGUGGAGCCCUCAGGCUCAdTdT | 43 | UGAGCCUGAGGGCUCCACCdTdT | 44 |
| 21-15# | | | | |
| HBV- | CAGUCAGGAAGGCAGCCUAdTdT | 45 | UAGGCUGCCUUCCUGACUGdTdT | 46 |
| 21-16# | | | | |
| HBV- | GGUGGCUCCAGUUCAGGAAdTdT | 47 | UUCCUGAACUGGAGCCACCdTdT | 48 |
| 21-17# | | | | |
| HBV- | GUUGACAAGAAUCCUCACAdTdT | 49 | UGUGAGGAUUCUUGUCAACdTdT | 50 |
| 21-18# | | | | |
| HBV- | CACAAGAAUCCUCACAAUAdTdT | 51 | UAUUGUGAGGAUUCUUGUCdTdT | 52 |
| 21-19# | | | | |
| HBV- | CUAGACUCGUGGUGGACUUdTdT | 53 | AAGUCCACCACGAGUCUAGdTdT | 54 |
| 21-20# | | | | |
| HBV- | GAGAUUAGGUUAAAGGUCUdTdT | 55 | AGACCUUUAACCUAAUCUCdTdT | 56 |
| 21-21# | | | | |
| HBV- | GCAUAAAUUGGUCUGCGCAdTdT | 57 | UGCGCAGACCAAUUUAUGCdTdT | 58 |
| 21-22# | | | | |
| HBV- | GCGCACCAGCACCAUGCAAdTdT | 59 | UUGCAUGGUGCUGGUGCGCdTdT | 60 |
| 21-23# | | | | |
| HBV- | CCUCUGOCUAAUCAUCUCUdTdT | 61 | AGAGAUGAUUAGGCAGAGGdTdT | 62 |
| 21-24# | | | | |
| HBV- | CACUUCCGGAAACUACUGUdTdT | 63 | ACAGUAGUUUCCGGAAGUGdTdT | 64 |
| 21-25# | | | | |
| HBV- | CCCUCAGGCUCAGGGCAUAdTdT | 65 | UAUGCCCUGAGCCUGAGGGdTdT | 66 |
| 21-26# | | | | |
| HBV- | CGAGGCAGGUCCCCUAGAAdTdT | 67 | UUCUAGGGGACCUGCCUCGdTdT | 68 |
| 21-27# | | | | |
| HBV- | GGGAAUCUCAAUGUUAGUAdTdT | 69 | UACUAACAUUGAGAUUCCCdTdT | 70 |
| 21-28# | | | | |
| HBV- | GGAAUCUCAAUGUUAGUAUdTdT | 71 | AUACUAACAUUGAGAUUCCdTdT | 72 |
| 21-29# | | | | |
| HBV- | CAUCACAUCAGGAUUCCUAdTdT | 73 | UAGGAAUCCUGAUGUGAUGdTdT | 74 |
| 21-30# | | | | |
| HBV- | GAACAUGGAGAACAUCACAUCAGGA | 75 | UCCUGAUGUGAUGUUCUCCAUGUUC | 76 |
| 25-8# | | | | |
| HBV- | GGAGAACAUCACAUCAGGAUUCCUA | 77 | UAGGAAUCCUGAUGUGAUGUUCUCC | 78 |
| 25-9# | | | | |
| HBV- | GAACAUCACAUCAGGAUUCCUAGGA | 79 | UCCUAGGAAUCCUGAUGUGAUGUUC | 80 |
| 25-10# | | | | |
| HBV- | CUUGUUGACAAGAAUCCUCACAAUA | 81 | UAUUGUGAGGAUUCUUGUCAACAAG | 82 |
| 25-11# | | | | |
| HBV- | CAGAGUCUAGACUCGUGGUGGACUU | 83 | AAGUCCACCACGAGUCUAGACUCUG | 84 |
| 25-12# | | | | |
| HBV- | GAGUCUAGACUCGUGGUGGACUUCU | 85 | AGAAGUCCACCACGAGUCUAGACUC | 86 |
| 25-13# | | | | |
| HBV- | GUCUAGACUCGUGGUGGACUUCUCU | 87 | AGAGAAGUCCACCACGAGUCUAGAC | 88 |
| 25-14# | | | | |
| HBV- | GCAUGGAGACCACCGUGAACGOCCA | 89 | UGGGCGUUCACGGUGGUCUCCAUGC | 90 |
| 25-15# | | | | |
| HBV- | CUGUAGGCAUAAAUUGGUCUGCGCA | 91 | UGCGCAGACCAAUUUAUGCCUACAG | 92 |
| 25-16# | | | | |
| HBV- | GCAUAAAUUGGUCUGCGCACCAGCA | 93 | UGCUGGUGCGCAGACCAAUUUAUGC | 94 |
| 25-17# | | | | |
| HBV- | GCCUUGGGUGGCUUUGGGGCAUGGA | 95 | UCCAUGCCCCAAAGCCACCCAAGGC | 96 |
| 25-18# | | | | |
| HBV- | GAAUUUGGAGCUACUGUGGAGUUAC | 97 | GUAACUCCACAGUAGCUCCAAAUUC | 98 |
| 25-19# | | | | |
| HBV- | CUCCAGUUCAGGAACAGUAAACCCU | 99 | AGGGUUUACUGUUCCUGAACUGGAG | 100 |
| 25-20# | | | | |
| HBV- | CCUGAGCAUUGCUCACCUCACCAUA | 101 | UAUGGUGAGGUGAGCAAUGCUCAGG | 102 |
| 25-21# | | | | |
| HBV- | CCACCAAUCGGCAGUCAGGAAGGCA | 103 | UGCCUUCCUGACUGCCGAUUGGUGG | 104 |
| 25-22# | | | | |

These siRNA sequences were designed based on the HBV genome according to Genbank ID:NC_003977.2, KY003230.1, AB933282.1, KR013949.1, KR014081.1, AF090840.1, AF325900.1, U95551.1, X02763.1, X70185.1, and X75311.1.

As described below in the Examples, these sequences were tested for the ability to inhibit viral gene expression and viral replication in in vitro assays. Based on these assays, some siRNAs were selected for modification to improve their activity and stability. The sequences of exemplary modified siRNA are shown below. For each siRNA pair the sense strand is shown first and the antisense strand second, and each sequence is provided in the 5′-3′ direction. In these molecules “m” prior to a base indicates a 2′-OMe modification and “f” indicates a 2′F modification. “P” indicates a phosphate moiety

HBV-21-3# mod
(SEQ ID NO: 105)
mCmGmCmAmGmUfCfCfCmCmAmAmCmCmUmCmCmAmAdTdT
(SEQ ID NO: 106)
PmUfUmGmGmAmGmGmUmUmGmGmGmGfAmCmUmGmCmGdTdT
HBV-21-6# mod
(SEQ ID NO: 107)
mCmCmUmCmUmUfCfAfUmCmCmUmGmCmUmGmCmUmAdTdT
(SEQ ID NO: 108)
PmUfAmGmCmAmGmCmAmGmGmAmUmGfAmAmGmAmGmGdTdT
HBV-21-9# mod
(SEQ ID NO: 109)
mCmUmCmUmCmGfUfCfCmCmCmUmUmCmUmCmCmGmUdTdT
(SEQ ID NO: 110)
PmAfCmGmGmAmGmAmAmGmGmGmGmAfCmGmAmGmAmGdTdT
HBV-21-11# mod
(SEQ ID NO: 111)
mCmCmUmAmUmUfGfAfUmUmGmGmAmAmAmGmUmAmUdTdT
(SEQ ID NO: 112)
PmAfUmAmCmUmUmUmCmCmAmAmUmCfAmAmUmAmGmGdTdT
HBV-21-22# mod
(SEQ ID NO: 113)
mGmCmAmUmAmAfAfUfUmGmGmUmCmUmGmCmGmCmAdTdT
(SEQ ID NO: 114)
PmUfGmCmGmCmAmGmAmCmCmAmAmUfUmUmAmUmGmCdTdT
HBV-25-3# mod
(SEQ ID NO: 115)
mCmCmAmAmCmCmUmCmCmAmAmUfCfAfCmUmCmAmCmCmAmAmCmCmU
(SEQ ID NO: 116)
PmAfGmGmUmUmGmGmUmGmAmGmUmGfAmUmUmGmGmAmGmGmUmUmGm
G
HBV-25-16# mod
(SEQ ID NO: 117)
mCmUmGmUmAmGmGmCmAmUmAmAfAfUfUmGmGmUmCmUmGmCmGmCmA
(SEQ ID NO: 118)
PmUfGmCmGmCmAmGmAmCmCmAmAmUfUmUmAmUmGmCmCmUmAmCmAm
G

### Targeting Ligands

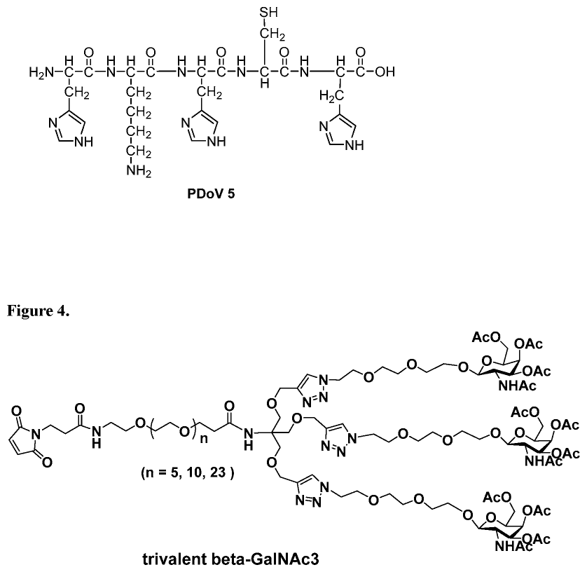

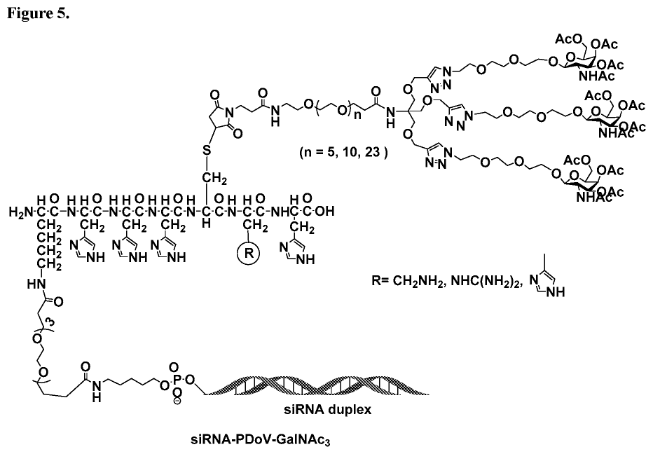

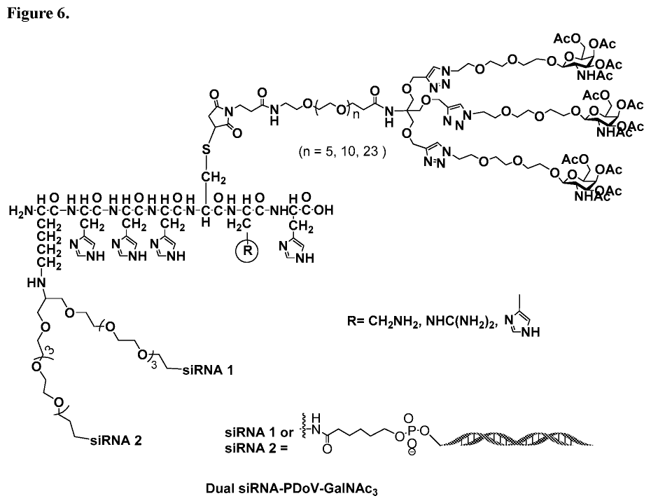

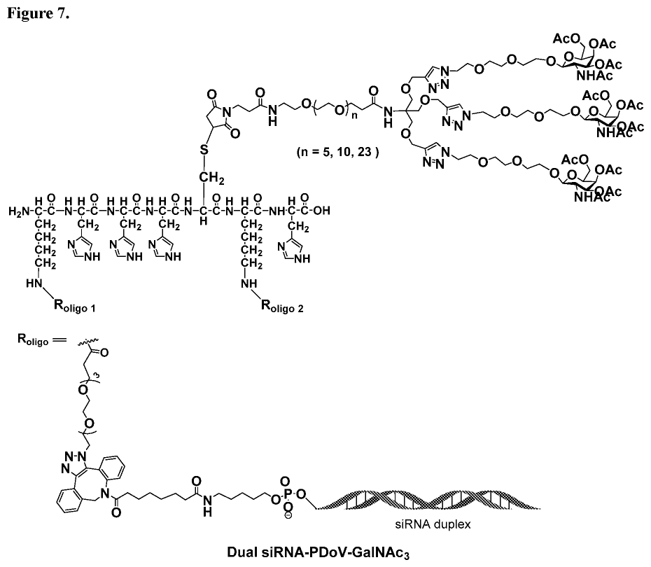

The targeting ligand moiety disclosed here is N-acetyl-galactosamine (GalNAc), galactose, galactosamine, N-formyl-galactosoamine, N-propionyl-galactosamine, or N-butanoylgalactosamine The targeting ligands were covalently coupled to the peptide. One, two or three targeting ligands may be used, and may have a structure as represented in FIG. 4. In that structure, coupling to the remained of the PDoV is achieved via reaction between a cysteine residue in the peptide and the maleimide on the targeting ligand.

### Endosome Releasing Docking Peptide

The Peptide Docking Vehicle (PDoV) advantageously has one ligand conjugation site together with multiple oligonucleotide sites. The PDoV has a peptide backbone with the general structure: (HₙKₘ)ₒXₚY_{q} (SEQ ID NO: 136) with multiple repeating units of histidine (H), lysine (K) and functional units X and Y (where X or Y is an amino acid, or an amino acid derivative), and where: n=1-10; m=1-10; o=1-10, p=1-5, and q=1-5. HK repeating units have been demonstrated to facilitate endosome release. The lysine residues or the functional unit(s) X may be used as docking sites for the conjugation of ligands and Y provides docking sites for the conjugation of oligonucleotide via a different covalent linkage. FIGS. 1 and 2 show a schematic of how the PDoV may be conjugated. For example, site \textcircled{1} is only able to react in the presence of ligand such as GalNAc or other targeting ligands. Site \textcircled{3} can only conjugate with oligonucleotide and siRNA under selected conditions. The differential reaction between the sites can be achieved by exploiting the different reactivities of, for example, amino and sulfhydryl moieties on amino acid side chains of the peptide. Methods for selective reactions between amino and sulfhydryl moieties are well known in the art.

### Administration

Suitably formulated compositions of the disclosed embodiments can be administered by means known in the art such as by parenteral routes, including intravenous, intramuscular, intraperitoneal, subcutaneous, subdermal, transdermal, airway (aerosol), rectal, vaginal and topical (including buccal and sublingual) administration. In some embodiments, the pharmaceutical compositions are administered by intravenous or intra-parenteral infusion or injection. In one embodiment, the composition is administered by injection into the tissue. In another embodiment, the composition is ministered by subcutaneous injection into a mammal. In still another embodiment, the composition is administered topically to the mammal.

A formulation is prepared to be compatible with its intended route of administration. Examples of routes of administration include parenteral, e.g., intravenous, intradermal, subcutaneous, subdermal, oral (e.g., inhalation), transdermal (topical), transmucosal, and rectal administration. Solutions or suspensions used for parenteral, intradermal, subdermal or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The siRNA formulations can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996). Further, the siRNA formulations can also be administered by a method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

The disclosed embodiments are further illustrated by the examples below, which are non-limiting.

### EXAMPLES

### Example 1

### Target Selection Based on HBV Genome Structure and Protein Function

Conserved sequences were chosen as candidate regions for siRNA design so at to obtain siRNA molecules that can inhibit all types of HBV strains. Eleven strains of HBV (NCBI accession numbers NC_003977.2,KY003230.1, AB933282.1, KR013949.1, KR014081.1, AF090840.1, AF325900.1, U95551.1, X02763.1, X70185.1, and X75311.1) were used for the siRNA molecules design. These strains were chosen as they can cover as many mutants of HBV as possible.

### Example 2

### Design of siRNAs Targeting Critical Genes of HBV

The P ORF codes for the reverse transcriptase domain of HBV polymerase that represents the target of antiviral agents such as nucleoside/nucleotide analogues and acyclic nucleotide analogs. Due to the overlapping S reading frame, inhibiting the reverse transcriptase domain can also knock down the S ORF.

The S ORF encodes three envelope proteins (large, middle and small) which are determinant for virus assembly and virus attachment to hepatocytes. Large protein is the substrate for viral receptor attachment; Middle protein function is not well understood and the Small protein is commonly referred to as the HBsAg or Australian antigen. The small, middle and the large proteins are detected as HBsAg. S region is the hepatocyte binding site and is also associated with occult HBV status. Moreover, the S region can influence the expression, synthesis, and secretion of HBsAg. The S deletions cannot lead to the progression of liver disease.

The C ORF encodes for two proteins, one structural, the HBcAg, that forms the nucleocapsid, and the HBeAg that is a secretion protein. HBeAg is the marker of HBV replication and infectivity. In the natural course of HBV chronic infection, the loss of HBeAg expression and the appearance of antibodies directed against it (anti-HBe) usually represent the end of viral replication and the resolution of hepatitis. Mutations in the pre-core and core regions cause HBeAg-negative chronic hepatitis B with presence of anti-HBe, in which replicative infection continues and HBV-DNA remains detectable.

The X ORF encodes for a multifunctional nonstructural protein whose functions are still unclear. It has been proposed to play a role in the establishment of infection and viral replication. A role for gene X in HBV carcinogenesis has been recently hypothesized.

The rationale behind the design of the siRNAs was for the sequences to cover as many mutants of HBV among the 7 selected HBV strains. Two lengths for the siRNA molecules were selected: 21 mer (19+dTdT) and 25 mer.

The siRNA molecules as described herein inhibit the expression of HBV nucleic acid sequences. Preferably, the HBV target gene is the polymerase (P) gene. Accordingly, in one embodiment, the siRNA molecules inhibit expression of one or more target sequences in the HBV P gene. The HBV genome has overlapping open reading frames. As such, targeting particular sequences of the polymerase gene will also target the same sequences in the overlapping gene. The siRNA molecules of the invention therefore are capable of targeting multiple genes with a single effector sequence. In each preferred embodiment, however, at least the polymerase gene is targeted. Because the genes of the HBV genome, in most regions, overlap among the four ORFs the siRNA molecules of the invention can also target the C gene, S gene, and X gene of the HBV.

### Example 3

### Conjugation of the siRNA and Multivalent GaINAc.

Conjugation of the siRNA and Group 3 (multivalent GalNAc-linker) of the PDoV can be via the 2′ and 5′ nucleotide position, or via the 5′ and 2′ nucleotide position as shown below:

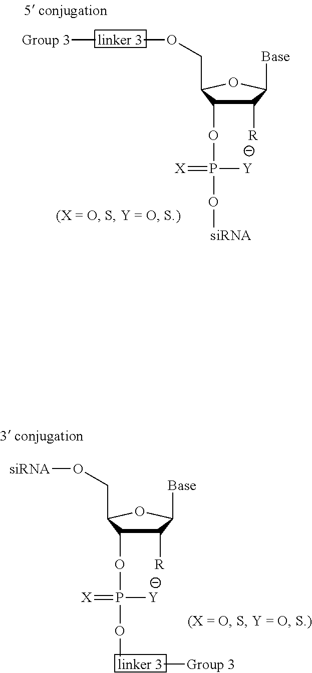

### Example 4

### Cell Culture Based Screening for Potent Anti-HBV siRNA Molecules

To identify the most potent siRNA for silencing HBV genes in A549 and 293T culture experiments, a recombinant psiCHECK-2 plasmid with DNA fragments carrying HBV gene sequences was used. HepG2 cells infected with real HBV were used to test the selected siRNA for their anti-HBV infecting activities.

To investigate the effect of siRNA candidates in degrading targeted HBV genes, a dual luciferase reporter vector, psiCHECK-2, with gene fragments of Surface, Core,E, X, polymerase, reverse transcriptase genes was used. psiCHECK-2 vectors are designed to provide a quantitative and rapid approach for initial optimization of RNA interference (RNAi). The vectors enable monitoring of changes in expression of a target gene fused to a reporter gene. The DNA fragments of HBV genome were synthesized, and then cloned into the multiple cloning sites of the psiCHECK-2 vector. In this vector, Renilla luciferase is used as a primary reporter gene, and the siRNA targeting genes are located downstream of the Renilla translational stop codon.

A549 or 293T cells were seeded in 96-well plates and incubated for 12 h. The reporter plasmids (recombinant vectors) psi-P&S&X, psi-P&X&C&CO uORF, and siRNA candidates were co-transfected into A549 or 293T cells using Lipofectamine 2000 in DMEM without FBS. The blank psi vector is taken as a negative control. Six hours post-transfection, the culture medium was replaced with DMEM supplemented with 10% FBS. 18, 24, 36 and 48 h post-transfection the activity of firefly luminescence and Renilla luciferase in each well was detected using the Dual Luciferase Kit. The siRNA candidates dramatically decreased luciferase activity, indicating that the siRNAs could greatly inhibit the expression of the target genes of HBV. These candidates were selected for the assay of infection with HBV in vitro.

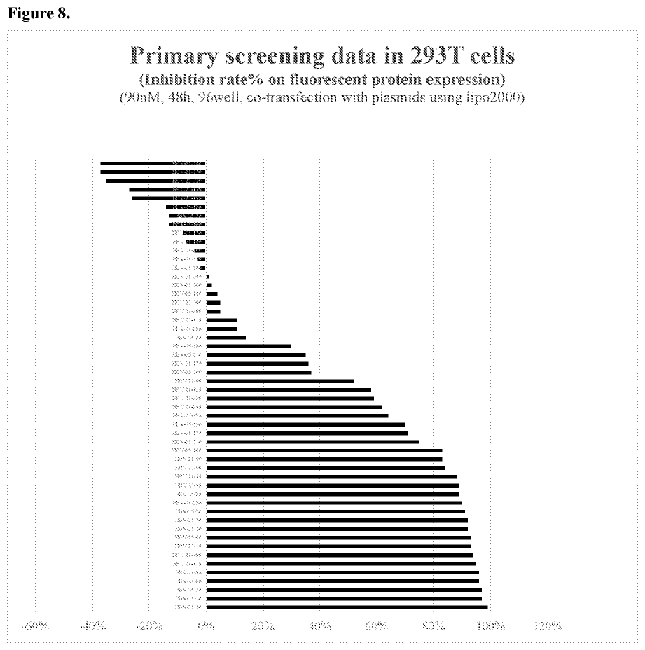

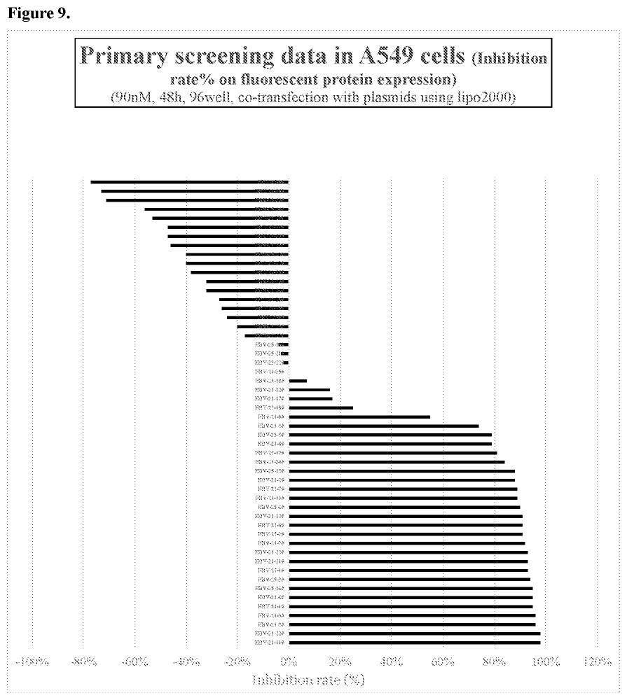

Through the above-mentioned fluorescence-based quantitative expression analysis method, the effect of the designed siRNAs on the inhibition of target genes expression in two kinds of cells was measured (A549 or 293T, see FIG. 8 and FIG. 9). The results showed that multiple siRNAs could inhibit the expression of target genes, including Seq. 01 (NTS00042-21-1#), Seq. 03 (NTS00042-21-3#), Seq. 06 (NTS00042-21-6#), Seq. 07 (NTS00042-21-7#), Seq. 09 (NTS00042-21-9#), Seq. 10 (NTS00042-21-10#), Seq. 11 (NTS00042-21-11#), Seq. 22 (NTS00042-21-22#), Seq. 31 (NTS00042-25-1#), Seq. 32 (NTS00042-25-2#), Seq. 33 (NTS00042-25-3#), Seq. 34 (NTS00042-25-4#), Seq. 36 (NTS00042-25-6#), Seq. 37 (NTS00042-25-7#), Seq. 46 (NTS00042-25-16#) and so on. The siRNA with the best inhibitory effect, such as Seq. 01 (NTS00042-21-1#), Seq. 03 (NTS00042-21-3#), Seq. 11 (NTS00042-21-11#), and Seq. 33 (NTS00042-25-3#), have significant inhibitory effects on target genes by more than 90% or even 95%.

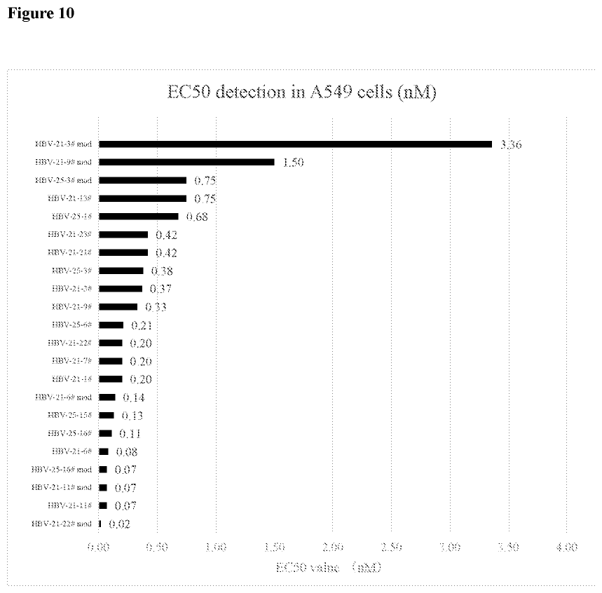

Several of the siRNA molecules, including some modified molecules as described above, were then used at varying concentrations in the same to determine EC50 values. The results are shown in FIG. 10.

### Example 5

### Sscreening in an HBV Cell Level Model (HepAD38) Cells

HepAD38 is a cell line that replicates human hepatitis B virus (HBV) under conditions that can be regulated with tetracycline (TET). In the presence of the antibiotic, this cell line is free of virus due to the repression of pregenomic (pg) RNA synthesis. When tetracycline is removed from the culture medium, the cells express viral pg RNA, accumulate subviral particles in the cytoplasm that contain DNA intermediates characteristic of viral replication, and secrete virus-like particles into the supernatant.

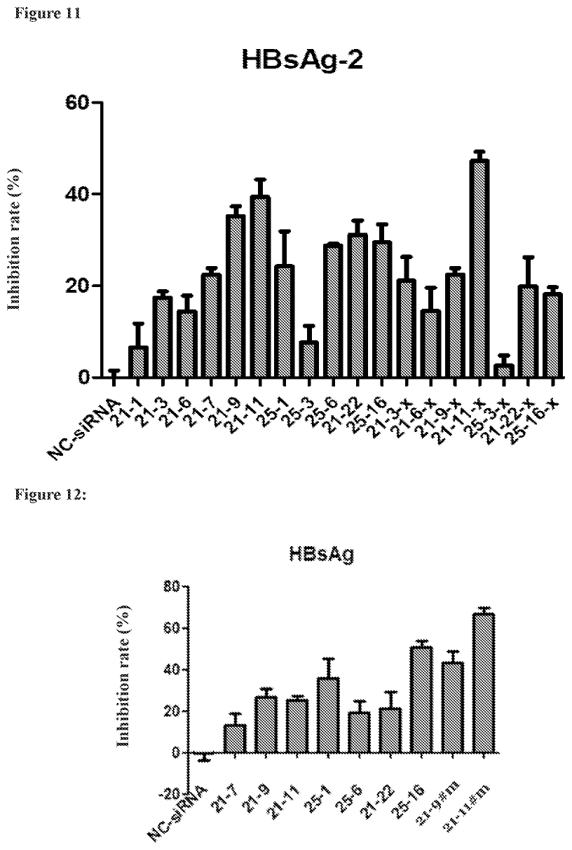

In this assay a culture of HepAD38 is activated by removal of TET, siRNA is transfected into the cell as described above, and after 7 days the cells and cell supernatant are collected. The following tests were carried out: ELISA test for HBV, HBsAg, and HBeAg; q-PCR test of the supernatant for HBV core particles; q-PCR test of the cytoplasm for HBV core particles; and Q-PCR test of the cell nucleus for HBV cccDNA. The siRNAs used in an initial screen were: #; HBV-21-1#; HBV-21-3#; HBV-21-3# mod; HBV-21-6#; HBV-21-6190 mod; HBV-21-7#; HBV-21-9#; HBV-21-9# mod; HBV-21-11#; HBV-21-11# mod; HBV-21-22#; HBV-21-22# mod; HBV-25-1#; HBV-25-3#; HBV-25-3# mod; HBV-25-6#; HBV-25-16#; and HBV-25-16# mod. The results of an initial screen 96h after transfection are shown in FIG. 11, where the y-axis measures % inhibition of HBsAG-2 production.

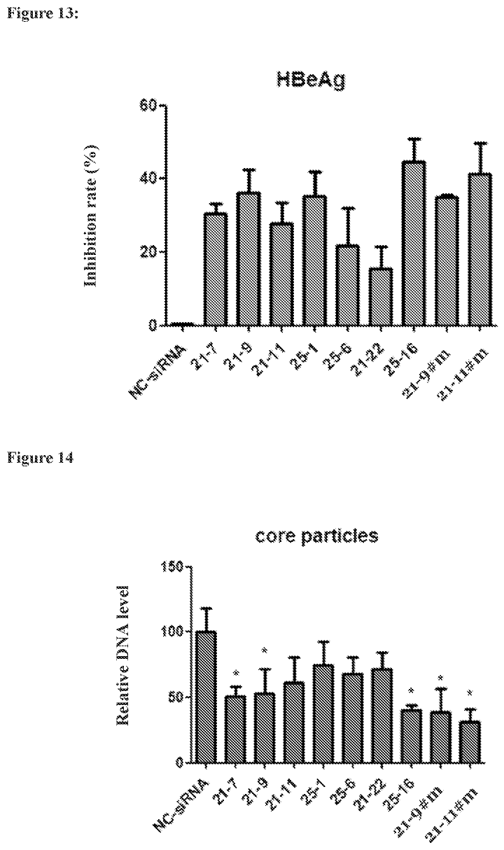

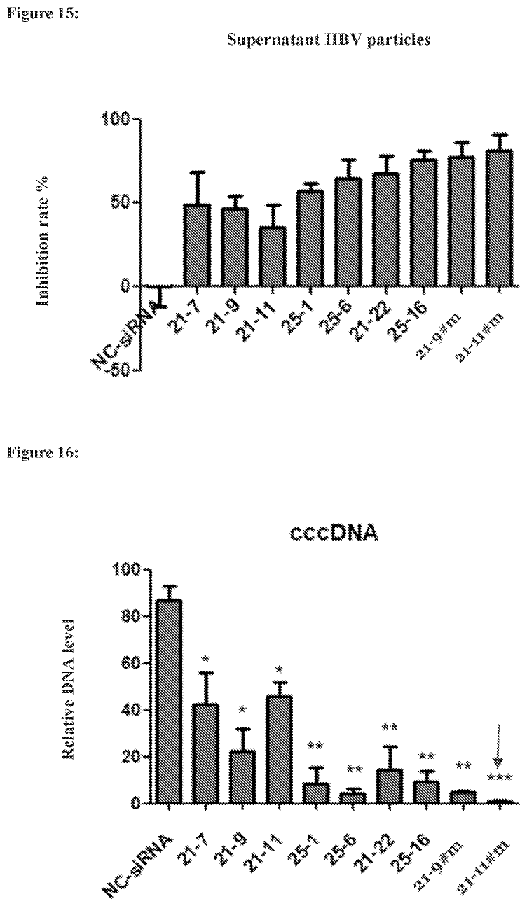

Several siRNAs were then assessed in a second round of screening. The siRNAs used in this assay were: 21-7#; 21-9#; 21-11#; 21-22#; 25-1#; 25-6#; 25-16#; 21-9#mod; and 21-11#mod. The results observed 7 days after transfection are shown in FIGS. 12-16. These results show the excellent effects of the siRNA molecules against HBV replication.

Although this disclosure describes certain embodiments of the compositions and methods, and many details have been set forth for purposes of illustration, it will be apparent to those skilled in the art that these compositions and methods are susceptible to additional embodiments and that certain of the details described herein may be varied without departing from the basic principles of the disclosure.

1. A pharmaceutical construct comprising a Peptide Docking Vehicle (PDoV) covalently linked to: (a) a targeting moiety; and (b) a first therapeutic nucleic acid, wherein said therapeutic nucleic acid inhibits replication of Hepatitis B virus (HBV).

2. The construct of claim 1, wherein the therapeutic nucleic acid is an siRNA molecule having a sequence selected from the group consisting of SEQ ID Nos. 101-118.

3. The construct of claim 2, further comprising a second siRNA molecule that is the same or different from the first siRNA molecule.

4. The construct of claim 1, wherein said PDoV has structure I or II, wherein A and B are independently a peptide sequence of H, K, R, HH, HHH, HHRH (SEQ ID NO: 120), HHK, HHHK (SEQ ID NO: 121) or any other endosomal releasing short peptide, D is an siRNA, R_{L} is a targeting ligand, and R_{S} is a covalent linker to the nucleic acid 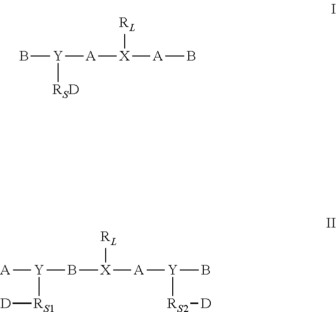
wherein the Type X sites are used to conjugate the targeting ligands, and the Type Y sites are used to conjugate the oligonucleotide.

5. The construct of claim 4 wherein the PDoV peptide construct has a structure selected from the group consisting of PDoV 1-5 (SEQ ID NOS 127-131, respectively, in order of appearance):

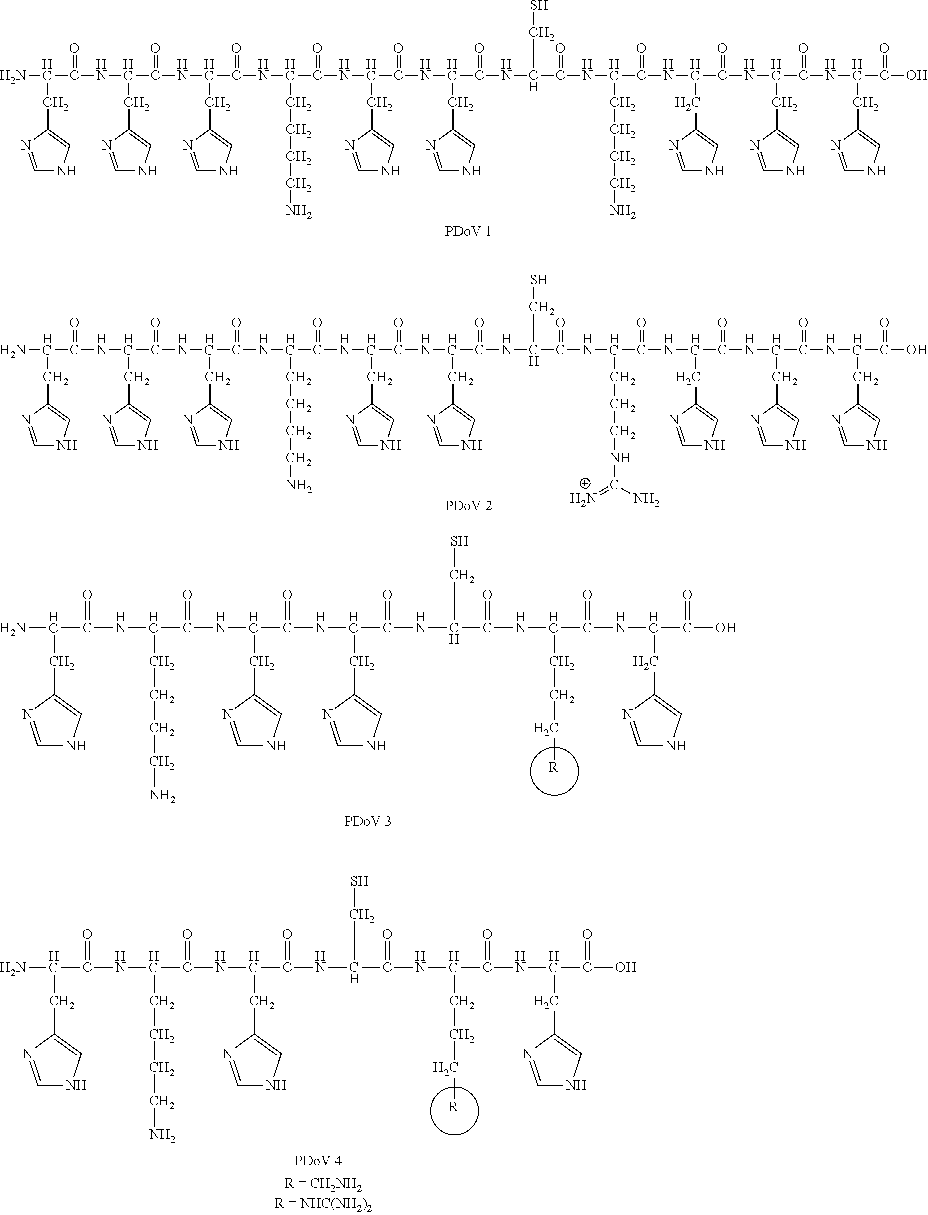

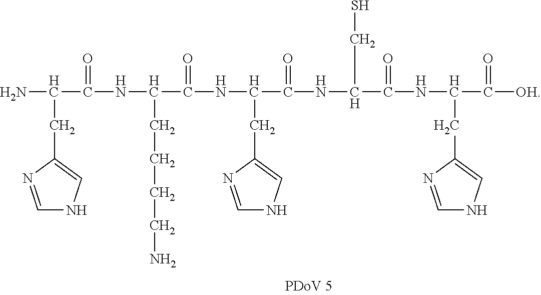 [SMILES: NCCCCC(NC(=O)C(N)Cc1c[nH]cn1)C(=O)NC(Cc1c[nH]cn1)C(=O)NC(CS)C(=O)NC(Cc1c[nH]cn1)C(=O)O]

6. The construct of claim 1, wherein said targeting moiety comprises a ligand covalently linked to said PDoV via a linker of formula III or IV, wherein n is 1-3: 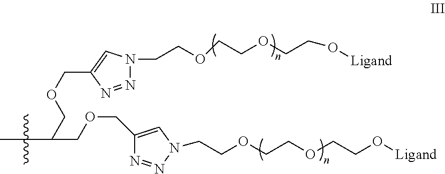
wherein n is 1, 2, or 3 and is connected to the dipodal linkage through a 1, 5-triazol ring with a CH₂OCH₂ unit; or 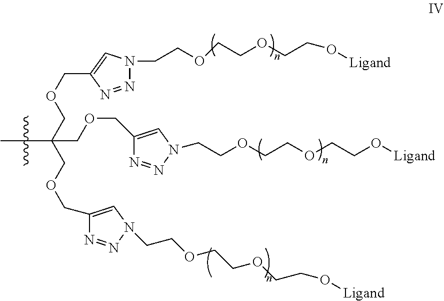
wherein n is 1, 2, or 3 and is connected to the tripodal linkage through a 1, 5-triazol ring with a CH₂OCH₂ unit.

7. The construct of claim 1, wherein the linker between the targeting moiety and the PDoV peptide comprises a polyethylene glycol chain —(CH₂CH₂O)ₙ—, or an alkylene chain —(CH₂CH₂)ₙ— chain, wherein n is an integer from 2-15.

8. The construct of claim 4, wherein R_{S} is a bioorthogonal reactive moiety to conjugate the nucleic acid with said PDoV peptide, wherein the reactive moiety is selected from the group consisting of an amine, hydrazine, N-hydroxysuccinimide, azido, alkyne, carboxylic acid, thiol, maleimide, phosphine diester, or a chemical reactive moiety selected from the group consisting of:

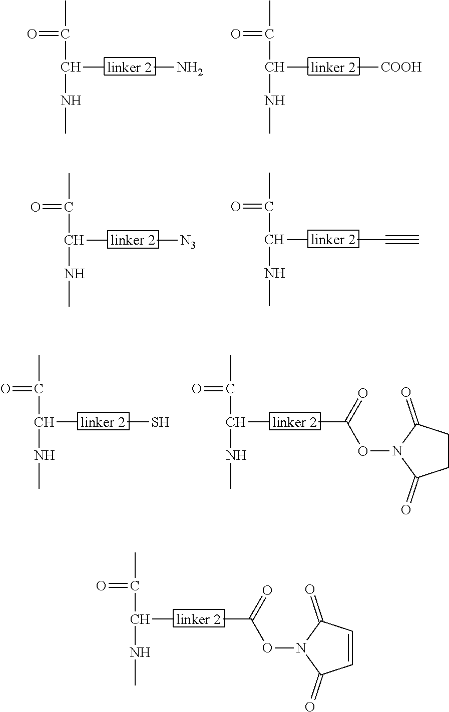

9. The construct of claim 2 wherein said siRNA molecule comprising at least one nucleotide chemically modified at the 2′ position, wherein the chemically modified nucleotide is selected from the group consisting of 2′-O-methyl, 2′-fluoro, 2′-O-methoxyethyl and 2′-O-allyl:

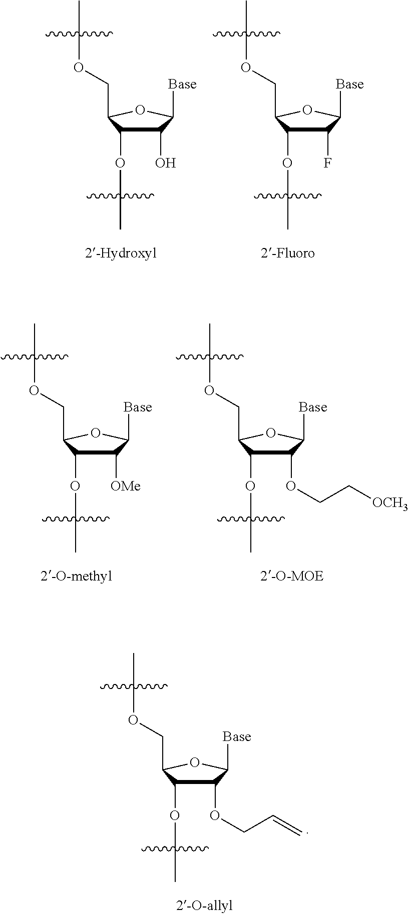

10. The construct of claim 1 wherein said siRNA molecule comprises one or more chemically modified nucleotides selected from the group consisting of a phosphorothioate diester, phosphorodithioate diester, and a phosphoronitro diester.

11. The construct of claim 1, wherein the therapeutic nucleic acid is an siRNA that targets the HBV S gene, wherein said siRNA has a sense strand consisting of the sequence CCUGCUGGUGGCUCCAGUUdTdT (SEQ ID No. 37) and an antisense strand consisting of the sequence AACUGGAGCCACCAGCAGGdTdT (SEQ ID No. 38).

12. The construct of claim 11 further comprising a second siRNA molecule that targets the HBV S gene.

13. The construct of claim 12, where each siRNA molecule has a sequence selected from the group consisting of SEQ ID Nos. 1-104.

14. The construct of claim 1 wherein the therapeutic nucleic acid is covalently linked to said PDoV via the 5′ or 3′ position of a nucleotide or nucleoside in said nucleic acid.

15. The construct of claim 14, wherein the linker is an aliphatic chain, a polyethylene glycol chain, or a hydrophobic or hydrophilic chain.

16. The construct of claim 1, wherein the targeting ligand is selected from the group consisting of N-acetyl-galactosamine (GalNAc), galactose, galactosamine, N-formal-galactosamine, N-propionyl-galactosamine, and N-butanoylgalactosamine.

17. The construct of claim 16, wherein the targeting ligand is N-acetyl-galactosamine (GalNAc).

18. A construct according to claim 1, wherein the PDoV comprises a C-terminal sequence comprising a C-terminal cysteine, and
wherein said C-terminal sequence comprises the sequence [KHHHKHHHHnKHHHKHHHK]₂KXC (SEQ ID NO: 122) where n=0 or 1 and wherein X is a synthetic molecule linker (C6H13, —(CH₂CH₂O)ₙ-linker, n=2-12) or a peptide linker selected from the group consisting of (serine, SSS, SSSS (SEQ ID NO: 123), SSSSS (SEQ ID NO: 124), SSSSSS (SEQ ID NO: 125), and TTTT (SEQ ID NO: 126)) between the terminal cysteine and a therapeutic molecule.

19. The construct of claim 18, wherein said therapeutic molecule is selected from the group consisting of lamivudine, adefovir, entecavir, telbivudine, and tenofovir.

20. A construct according to claim 1 having a structure selected from the group consisting of (SEQ ID NOS 132-134, respectively, in order of appearance):

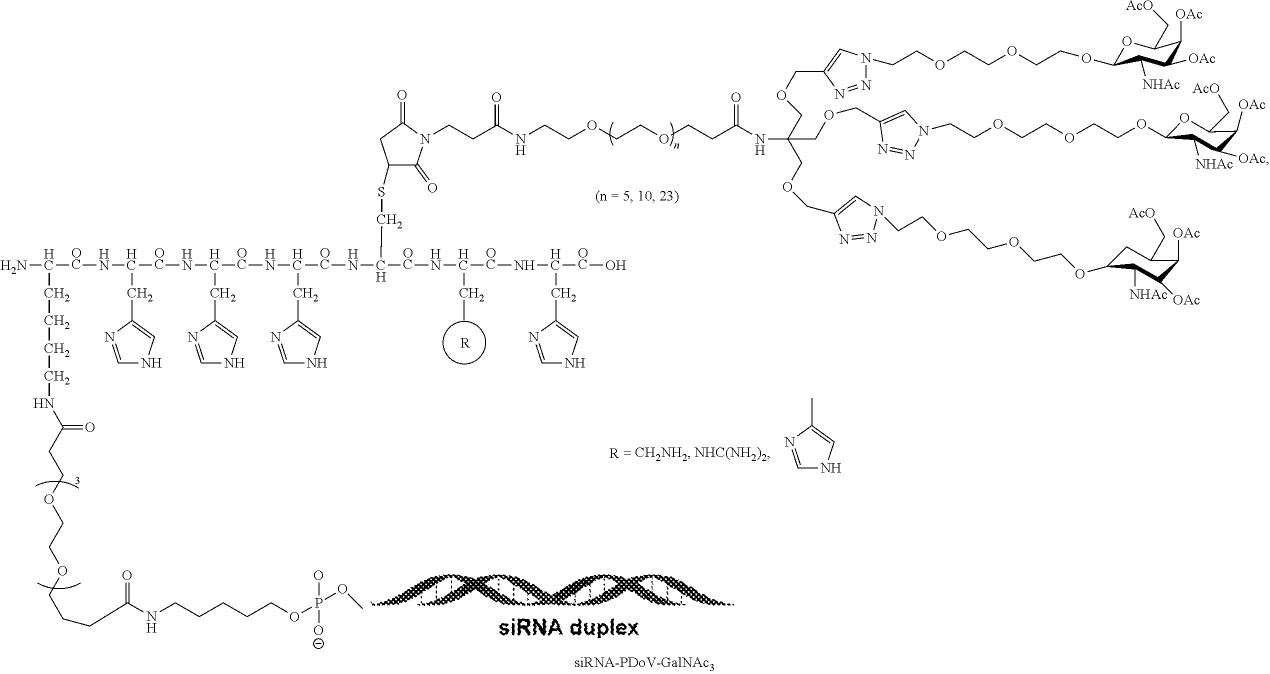

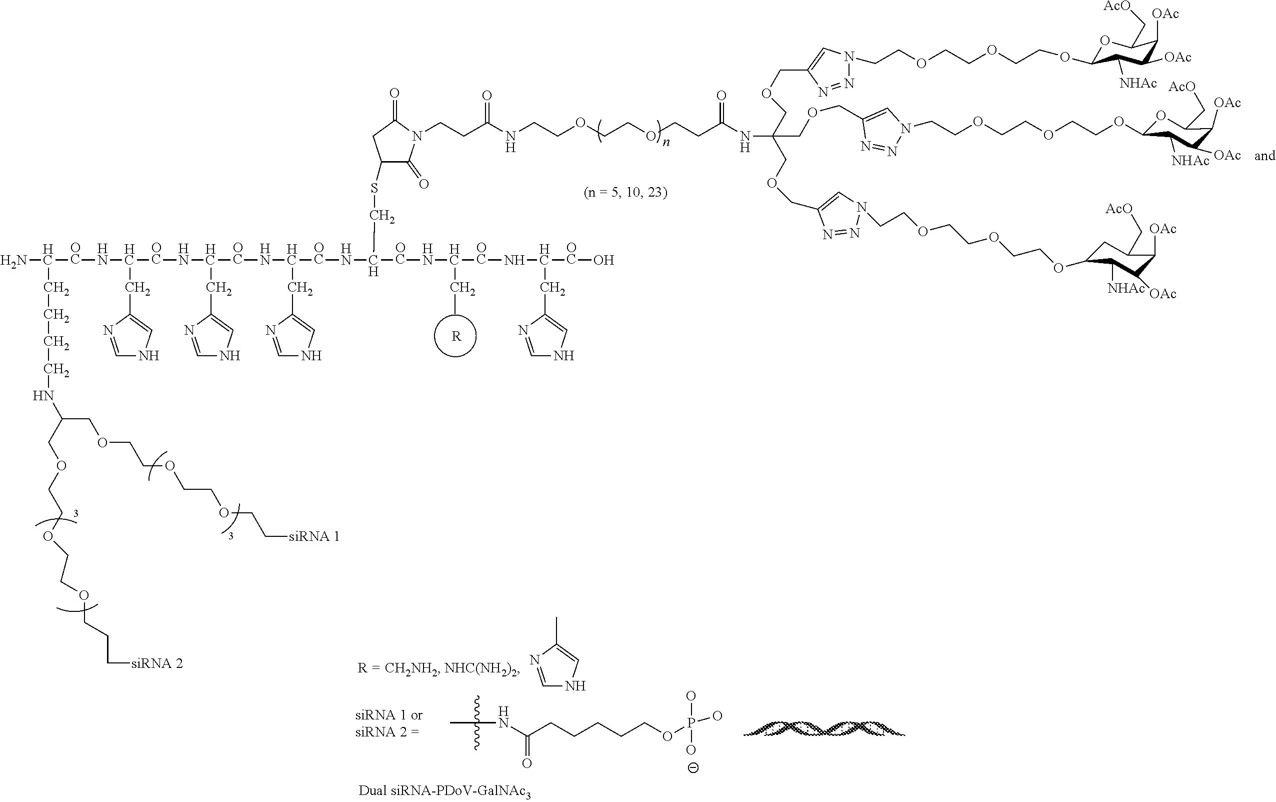

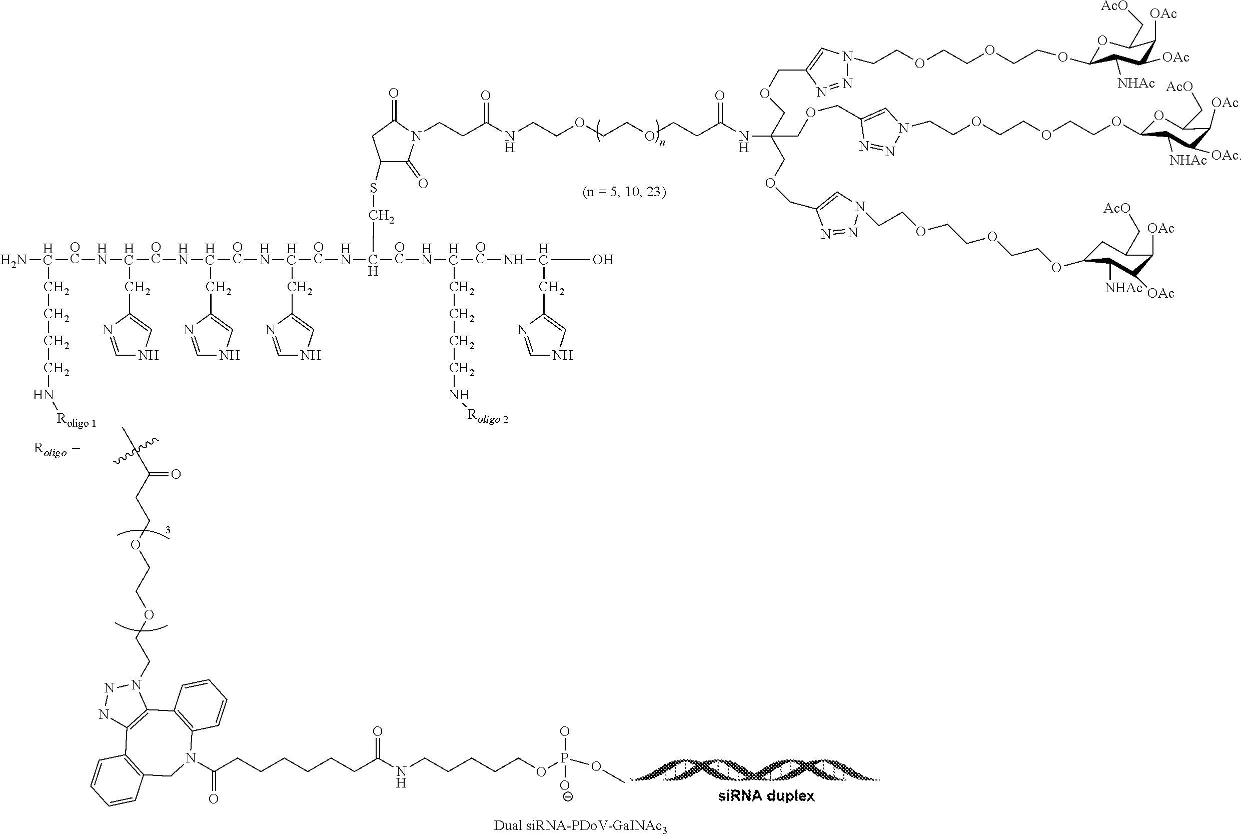

21. A pharmaceutical composition comprising a construct according to claim 1 and a pharmaceutically acceptable carrier.

22. A method of treating HBV in a subject, comprising administering to the described subject a pharmaceutical composition according to claim 21, wherein the subject is a human subject.
